(19) United States
(12) Patent Application Publication
(10) Pub. No.: US 2026/0331243 A1
(43) Pub. Date: Oct. 22, 2026

(54) Systems And Methods For Ascertaining Cancer-Related Risks Resulting From Multi-Cancer Early Detection Tests Using Population-Level Classification Data

(71) Applicant: Grail, Inc., Menlo Park, CA (US)

(72) Inventor: Earl Hubbell, Palo Alto, CA (US)

(21) Appl. No.: 19/401,062

(22) Filed: Nov. 25, 2025

Related U.S. Application Data
(60) Provisional application No. 63/724,773, filed on Nov. 25, 2024.
(60) Provisional application No. 63/729,930, filed on Dec. 9, 2024.

(51) Int. Cl. G16H 15/00; G16H 50/20; G16H 50/30; G16H 50/70

(52) U.S. Cl. CPC G16H 15/00; G16H 50/20; G16H 50/30; G16H 50/70

(57) ABSTRACT

Systems and methods for ascertaining cancer-related risks resulting from multi-cancer early detection (MCED) tests administered to individuals using population-level classification data are disclosed. According to certain aspects, an electronic device may access a data structure reflecting a prevalence dataset, an incidence dataset, a dataset on instances of interval cancers, or some combination thereof. The electronic device may analyze a result of an MCED test administered to an individual in combination with the data structure to determine a set of risk levels of the individual developing one or more of the plurality of cancers over a specified time period, and generate a digital report for the individual which provides a comprehensive post-test analysis.

### CROSS-REFERENCE TO RELATED APPLICATIONS

The present application claims the benefit of and priority to U.S. Provisional Application No. 63/724,773 filed on Nov. 25, 2024, and U.S. Provisional Application No. 63/729,930 filed on Dec. 9, 2024, both of which are incorporated by reference.

### BACKGROUND

The present disclosure is directed to improvements in early cancer detection. More particularly, the present disclosure is directed to platforms and technologies for ascertaining cancer-related risks resulting from multi-cancer early detection (MCED) tests using population-level classification data, and conducting comprehensive post-test analyses.

Early detection of cancer plays a crucial role in improving patient outcomes and survival rates. Several early cancer detection methods are currently available on the market, including screening or diagnostic methods that use techniques such as cell-free DNA (cfDNA) analysis, which allows for the identification of cancer-associated genetic and epigenetic material circulating in the bloodstream. These tests are designed to identify the presence of malignancy or predisposition to cancer at an early stage, potentially providing individuals with a critical window of opportunity for intervention. However, despite the growing availability of such detection methods, the information provided by these tests is limited in its ability to offer detailed insights into an individual's cancer risks. In particular, existing detection technologies generally provide a binary or probabilistic indication of the presence of cancerous or precancerous cells, but they fall short of identifying specific risks tied to particular types of cancer. There is currently no mechanism for determining the likelihood of an individual developing specific types of cancer based on any detected biomarkers, nor is there a method for establishing timeframes in which such cancers may develop.

Furthermore, existing detection methods do not provide a reliable assessment of the severity or aggressiveness of the specific cancers that may be developing. This lack of granularity in the results presents a technical challenge in the field of early cancer detection, as it impedes the ability to tailor individualized treatment or preventative strategies based on the specific risks, timing, or severity of potential cancer development. This technical gap underscores the need for advanced detection technologies that can not only identify the presence of cancer but also convey critical, individualized insights regarding cancer type, progression, and severity, thereby empowering more effective clinical decision-making.

### SUMMARY

In an embodiment, a computer-implemented method of ascertaining cancer-related risks resulting from a cancer screening test administered to an individual using population-level classification data is provided. The computer-implemented method may include: accessing, by at least one computer processor, a data structure comprising one or more datasets selected from the group consisting of (i) a prevalence dataset indicating an initial prevalence of a plurality of individuals who have one or more of a plurality of cancers, (ii) an incidence dataset indicating an incidence of the plurality of individuals who are newly diagnosed with one or more of the plurality of cancers within a specified time period, and (iii) a third dataset indicating instances of the plurality of individuals being diagnosed with one or more of the plurality of cancers between screening tests; obtaining, by the at least one computer processor, a result of the cancer screening test administered to the individual, wherein the result of the cancer screening test indicates whether a cancer signal was detected; analyzing, by the at least one computer processor, the result of the cancer screening test in combination with the data structure to determine a set of risk levels of the individual developing one or more of the plurality of cancers over the specified time period, wherein the set of risk levels accounts for at least an age and a gender of the individual; and generating, by the at least one computer processor based on the set of risk levels, a digital report for the individual, wherein the digital report indicates (i) whether a cancer signal was detected, (ii) if a cancer signal was not detected, a risk level, of the set of risk levels, of the individual being diagnosed with at least one cancer, of the plurality of cancers, over the specified time period, and (iii) if a cancer signal was detected, and for each of at least some of the cancers of the plurality of cancers, an additional risk level, of the set of risk levels, that a screening test corresponding to that cancer would result in the individual being diagnosed with that cancer.

In another embodiment, a system for ascertaining cancer-related risks resulting from a cancer screening test administered to an individual using population-level classification data is provided. The system may include: a memory storing a set of computer-readable instructions and a data structure comprising one or more datasets selected from the group consisting of (i) a prevalence dataset indicating an initial prevalence of a plurality of individuals who have one or more of a plurality of cancers, (ii) an incidence dataset indicating an incidence of the plurality of individuals who are newly diagnosed with one or more of the plurality of cancers within a specified time period, and (iii) a third dataset indicating instances of the plurality of individuals being diagnosed with one or more of the plurality of cancers between screening tests; and one or more processors interfaced with the memory. The one or more processors may be configured to execute the set of computer-readable instructions to cause the one or more processors to: obtain a result of the cancer screening test administered to the individual, wherein the result of the cancer screening test indicates whether a cancer signal was detected, analyze the result of the cancer screening test in combination with the data structure to determine a set of risk levels of the individual developing one or more of the plurality of cancers over the specified time period, wherein the set of risk levels accounts for at least an age and a gender of the individual, and generate, based on the set of risk levels, a digital report for the individual, wherein the digital report indicates (i) whether a cancer signal was detected, (ii) if a cancer signal was not detected, a risk level, of the set of risk levels, of the individual being diagnosed with at least one cancer, of the plurality of cancers, over the specified time period, and (iii) if a cancer signal was detected, and for each of at least some of the cancers of the plurality of cancers, an additional risk level, of the set of risk levels, that a screening test corresponding to that cancer would result in the individual being diagnosed with that cancer.

In another embodiment, a non-transitory computer-readable storage medium configured to store instructions executable by one or more processors is provided. The instructions may include: instructions for accessing a data structure comprising one or more datasets selected from the group consisting of (i) a prevalence dataset indicating an initial prevalence of a plurality of individuals who have one or more of a plurality of cancers, (ii) an incidence dataset indicating an incidence of the plurality of individuals who are newly diagnosed with one or more of the plurality of cancers within a specified time period, and (iii) a third dataset indicating instances of the plurality of individuals being diagnosed with one or more of the plurality of cancers between screening tests; instructions for obtaining a result of a cancer screening test administered to an individual, wherein the result of the cancer screening test indicates whether a cancer signal was detected; instructions for analyzing the result of the cancer screening test in combination with the data structure to determine a set of risk levels of the individual developing one or more of the plurality of cancers over the specified time period, wherein the set of risk levels accounts for at least an age and a gender of the individual; and instructions for generating, based on the set of risk levels, a digital report for the individual, wherein the digital report indicates (i) whether a cancer signal was detected, (ii) if a cancer signal was not detected, a risk level, of the set of risk levels, of the individual being diagnosed with at least one cancer, of the plurality of cancers, over the specified time period, and (iii) if a cancer signal was detected, and for each of at least some of the cancers of the plurality of cancers, an additional risk level, of the set of risk levels, that a screening test corresponding to that cancer would result in the individual being diagnosed with that cancer.

Further, in an embodiment, a computer-implemented method in an electronic device of rendering a digital report for an individual related to a result of a cancer screening test administered to the individual is provided. The computer-implemented method may include: receiving, from a server computer by at least one computer processor of the electronic device, the digital report for the individual, wherein the result of the cancer screening test was analyzed in combination with a data structure to determine a set of risk levels, accounting for at least an age and a gender of the individual, of the individual developing one or more of a plurality of cancers over a specified time period, and wherein the data structure comprises one or more datasets selected from the group consisting of (i) a prevalence dataset indicating an initial prevalence of a plurality of individuals who have one or more of the plurality of cancers, (ii) an incidence dataset indicating an incidence of the plurality of individuals who are newly diagnosed with one or more of the plurality of cancers within the specified time period, and (iii) a third dataset indicating instances of the plurality of individuals being diagnosed with one or more of the plurality of cancers between screening tests; initiating, by the electronic device, an application executed by the at least one computer processor and configured to render the digital report; and rendering the digital report via a user interface of the electronic device and within an interface associated with the application, wherein the digital report that was rendered indicates (i) the result of the cancer screening test administered to the individual, and (ii) the set of risk levels. The user interface may include elements for inputting information on diagnostic results from a diagnostic workup performed on the individual, e.g., by a healthcare provider. The user interface may be configured to update the cancer prediction and/or the tailored recommendations based on the input information.

In an additional embodiment, a computer-implemented method in an electronic device of rendering a digital report for an individual related to a result of a cancer screening test administered to the individual is provided. The computer-implemented method may include: receiving, from a server computer by at least one computer processor of the electronic device, the digital report for the individual, wherein the result of the cancer screening test was analyzed in combination with a data structure to determine a set of risk levels, accounting for at least an age and a gender of the individual, of the individual developing one or more of a plurality of cancers over a specified time period, and wherein the data structure comprises one or more datasets selected from the group consisting of (i) a prevalence dataset indicating an initial prevalence of a plurality of individuals who have one or more of the plurality of cancers, (ii) an incidence dataset indicating an incidence of the plurality of individuals who are newly diagnosed with one or more of the plurality of cancers within the specified time period, and (iii) a third dataset indicating instances of the plurality of individuals being diagnosed with one or more of the plurality of cancers between screening tests; initiating, by the electronic device, an application executed by the at least one computer processor and configured to render the digital report; and rendering the digital report via a user interface of the electronic device and within an interface associated with the application, wherein the digital report that was rendered indicates (i) that a cancer signal from the cancer screening test was detected, and (ii) a cancer signal origin (CSO) for the cancer signal.

Further, in an embodiment, a computer-implemented method of generating a digital report resulting from a cancer screening test administered to an individual is provided. The computer-implemented method may include: obtaining, by at least one computer processor, a result of the cancer screening test administered to the individual, wherein the result of the cancer screening test indicates (i) that a cancer signal was detected, and (ii) a cancer signal origin (CSO) for the cancer signal; determining, by the at least one computer processor based on an inclusion of the CSO for the cancer signal within a stored mapping, whether to include a set of additional predictive information (API) in the digital report; and generating, by the at least one computer processor, the digital report for the individual, wherein the digital report indicates (i) that the cancer signal was detected, (ii) the CSO for the cancer signal, and (iii) if it was determined to include the set of API, the set of API.

Moreover, in an embodiment, a computer-implemented method of generating a digital report resulting from a cancer screening test administered to an individual is provided. The computer-implemented method may include: obtaining, by at least one computer processor, a result of the cancer screening test administered to the individual, wherein the result of the cancer screening test indicates that a cancer signal was not detected, wherein the result of the cancer screening test was analyzed in combination with a data structure to determine a set of risk levels, accounting for at least an age and a gender of the individual, of the individual developing one or more of a plurality of cancers over a specified time period, and wherein the data structure comprises one or more datasets selected from the group consisting of (i) a prevalence dataset indicating an initial prevalence of a plurality of individuals who have one or more of the plurality of cancers, (ii) an incidence dataset indicating an incidence of the plurality of individuals who are newly diagnosed with one or more of the plurality of cancers within the specified time period, and (iii) a third dataset indicating instances of the plurality of individuals being diagnosed with one or more of the plurality of cancers between screening tests; and generating, by the at least one computer processor, the digital report for the individual, wherein the digital report indicates that a cancer signal was not detected.

### BRIEF DESCRIPTION OF THE FIGURES

FIG. 1 depicts an overview of components and entities associated with the systems and methods, in accordance with some embodiments.

FIG. 2 depicts an example signal diagram detailing analyses of MCED test results and cancer-related data structures, in accordance with some embodiments.

FIGS. 3A-3E depict example digital reports, each indicating that a cancer signal was not detected for the tested individual and showing cancer-related risks for the tested individual, in accordance with some embodiments.

FIGS. 4A-4D depict example digital reports, each indicating that a cancer signal was detected for the tested individual and showing cancer-related risks for the tested individual, in accordance with some embodiments.

FIG. 5 illustrates an example flow diagram of ascertaining cancer-related risks resulting from a MCED test administered to an individual using population-level classification data, in accordance with some embodiments.

FIG. 6 is an example hardware diagram of an electronic device and a server configured to perform various functionalities, in accordance with some embodiments.

FIG. 7 is an example digital report associated with a cancer screening test taken by an individual, in accordance with some embodiments.

FIG. 8 illustrates an example flow diagram of generating a digital report resulting from a MCED test administered to an individual, in accordance with some embodiments.

FIG. 9 illustrates another example flow diagram of generating a digital report resulting from a MCED test administered to an individual, in accordance with some embodiments.

FIG. 10 illustrates a flow diagram describing tailored digital reporting, in accordance with some embodiments.

FIG. 11 illustrates a flow diagram describing updated digital reporting based on diagnostic results, in accordance with some embodiments.

FIG. 12 illustrates a flow diagram describing prescreening-informed cancer prediction, in accordance with some embodiments.

FIG. 13 illustrates one example digital report indicating cancer signal detected, in accordance with one or more embodiments.

FIG. 14 illustrates one example digital report indicating cancer signal not detected, in accordance with one or more embodiments.

### DETAILED DESCRIPTION

### Overview of Cancer Prediction

Early detection of a disease state (such as cancer) in subjects is important as it allows for earlier treatment and therefore a greater chance for survival. Sequencing of DNA fragments in cell-free (cf) DNA samples can be used to identify features that can be used for disease classification. For example, in cancer assessment, cell-free DNA based features (such as presence or absence of somatic variant, methylation status, or other genetic aberrations) from a blood sample can provide insight into whether a subject may have cancer, and further insight on what type of cancer the subject may have. Towards that end, this description includes systems and methods for analyzing cell-free DNA sequencing data for determining a subject's likelihood of having a disease.

The present embodiments may relate to advanced cancer detection technologies that are configured to assess cancer-related risks using one or more data structures reflecting population-level classification data, as well as convey critical, individualized insights regarding cancer type, progression, and severity. According to certain aspects, systems and methods may analyze results of multi-cancer early detection (MCED) tests in combination with these data structures to determine a set of cancer-related risk levels for individuals who take the MCED tests.

According to embodiments, an MCED test is an advanced screening tool designed to detect multiple types of cancers from a blood sample (e.g., from a single blood sample or single blood draw from an individual). For instance, by analyzing cfDNA in the blood for specific methylation patterns associated with various cancers, methylation-based MCED tests can identify whether a cancer signal is present. If a cancer signal is detected, the test may provide information about the Cancer Signal of Origin (CSO), which predicts the likely tissue or organ where the cancer signal may have originated. Accordingly, the result of an MCED test may include a binary outcome (i.e., cancer signal detected or not detected), and the potential origin of the cancer if detected. In some examples, the potential origin of cancer indicated by the MCED test is one or more solid or liquid cancerous tissues of origin. Such cancers or types of cancer can include breast cancer, uterine cancer, cervical cancer, ovarian cancer, bladder cancer, urothelial cancer of renal pelvis, renal cancer other than urothelial, prostate cancer, anorectal cancer, colorectal cancer, esophageal cancer, gastric cancer, hepatobiliary cancer arising from hepatocytes, hepatobiliary cancer arising from cells other than hepatocytes, pancreatic cancer, squamous cell cancer of the upper gastrointestinal tract, upper gastrointestinal cancer other than squamous, head and neck cancer, lung cancer, lung adenocarcinoma, small cell lung cancer, squamous cell lung cancer and cancer other than adenocarcinoma or small cell lung cancer, neuroendocrine cancer, melanoma, thyroid cancer, sarcoma, multiple myeloma, lymphoma, and leukemia. Such cancers or types of cancer can also include brain cancer, vulvar cancer, vaginal cancer, testicular cancer, mesothelioma of the pleura, mesothelioma of the peritoneum, and/or gallbladder cancer.

It is noted that while MCED tests will be used for ease of discussion herein, various types of cancer detection tests for screening and/or for diagnosing cancer can be contemplated with the present embodiments. Merely by way of example and as described further below, tests can include MCED (such as Grail's Galleri test, otherwise referred to herein as the Galleri test or Grail test), multi-cancer detection (MCD), low-dose computed tomography (LDCT), mammography, colonoscopy, fecal immunochemical test (FIT), stool-based testing, and/or other types of screening tests. Additionally, other types of samples can be used for cancer detection.

For instance, an individual's test sample may be a sample selected from the group consisting of blood, plasma, serum, urine, fecal, and saliva samples. Alternatively, the test sample may comprise a sample selected from the group consisting of whole blood, a blood fraction (e.g., white blood cells (WBCs)), a tissue biopsy, pleural fluid, pericardial fluid, cerebral spinal fluid, and peritoneal fluid.

The present embodiments are configured to build, maintain, and/or access a data structure or model using population-level data that is organized across different phases of detection, including for example, a prevalence round, one or more incidence rounds, and/or the detection of interval cancers. Generally, the prevalence round may refer to an initial screening, which identifies cancers that are already present but undiagnosed in a population. The incidence round(s) may focus on new cancers that develop between subsequent screenings after the prevalence round, representing new cases that were not previously detected. Interval cancers are those diagnosed clinically (i.e., through symptoms) between regular screening rounds, indicating cancers that were missed in the last screening.

The present embodiments may analyze a resulting data structure in combination with results of MCED tests and/or other screening tests taken by individuals to determine a set of risk levels of these individuals developing one or more cancers over a specified time period. Additionally, the present embodiments may, based on the analysis, generate digital reports for the individuals, where the digital reports may convey different information based on the results of the MCED tests as well as any of the determined set of risk levels.

There are several limitations in the current state of early cancer detection techniques that present technical challenges that impact the effectiveness, accuracy, and reliability of the techniques. In particular, MCED tests primarily provide a binary result indicating whether a cancer signal is detected. If a signal is detected, the test predicts the likely CSO, but it does not provide a quantitative risk assessment indicating the likelihood of having or developing cancer, the possible stage or severity of any cancer, or screening test recommendations. This limits the ability to inform clinical decision-making accurately, as clinicians need more granular data, such as the probability of cancer presence, estimated stage, and growth rate, to determine the urgency and type of follow-up investigations. Without this information, patients may be subjected to unnecessary invasive procedures, overdiagnosis, or delayed diagnosis due to insufficient prioritization. This makes the test less actionable in a clinical setting, reducing its potential benefit. To enhance clinical utility, MCED tests would benefit from integrating data analyses that can provide a set of risk levels that conveys the risk or likelihood of cancer presence and its potential aggressiveness. Integrating the data analyses with screening test results as described herein can also enhance personal decision-making for individuals in regard to pursuing cancer screenings over time. For example, an individual receiving a negative MCED test result where no cancer signal is detected can receive further custom information from the data analyses, such as relevant cancer risks and risk levels should the individual not return for a screening test in the next year. Such additional information can provide actionable items for the individual by encouraging individuals to screen regularly (e.g., once a year, or otherwise within a cadence as suggested by the risk levels over time from the data analyses).

While some current MCED tests have high overall specificity (e.g., over 99%), the sensitivity varies significantly across different cancer types. In particular, current MCED tests are more sensitive to certain cancers that release more detectable cfDNA into the bloodstream, such as pancreatic or ovarian cancers, but less effective for others, particularly those that release lower levels of cfDNA or have slower growth rates, such as some lymphomas or prostate cancers. This inconsistency in sensitivity means that certain cancers could be missed, especially in their early stages when they are most treatable. Further, a lack of sensitivity undermines the testing goal of early detection and could lead to false reassurance for patients, resulting in missed opportunities for early intervention. Incorporating data structures such as those discussed herein are able to better differentiate between cfDNA signals from different cancer types and improve detection rates for cancers that currently have lower sensitivity.

Further, MCED tests currently do not provide information on timeframes associated with cancer progression, screening test recommendations, or the expected severity of the detected cancer. That is, the results indicate the presence of cancer but do not provide insight into how aggressive the cancer might be or how quickly it could progress. Knowing the likely progression rate of cancer is critical for clinical decision-making, particularly in deciding between immediate intervention or other approaches. Integrating data analyses with MCED test results leads to enhanced capabilities to forecast timeframes and potential outcomes, which enable for more personalized and effective treatment and/or cancer screen planning.

Moreover, there is currently limited data on the longitudinal performance of MCED tests over time and across diverse populations with varying genetic, lifestyle, and environmental factors. That is, the effectiveness of MCED tests can differ based on demographic factors and clinical characteristics such as age, gender, ethnicity, and medical history. Without sufficient longitudinal data and studies across diverse populations, it is challenging to generalize the performance of the test or adapt its use for specific groups at different risk levels. Large-scale, diverse cohort studies and ongoing validation trials are needed to assess the longitudinal accuracy, effectiveness, and equity of MCED tests in real-world settings, however such studies and trials are complex and demanding, and pose multiple technical challenges.

In particular, recruiting and retaining a large, diverse cohort of participants from different demographic backgrounds and clinical characteristics (e.g., age, gender, ethnicity, genetics, lifestyle) is challenging, time consuming, experiences high dropout rates, and requires certain ethical and regulatory approvals. Further, conducting extensive cohort studies and validation trials over long periods requires significant financial investment and resources, including advanced data infrastructure (e.g., secure databases, bioinformatics tools) to manage and analyze vast amounts of data collected over time, which also require continual updates and maintenance. Validation trials also require regular follow-ups with participants to confirm test results, which often involve additional diagnostic tests like imaging or biopsies, thus adding to the overall time and resource commitment.

Additionally, collecting longitudinal data on the performance of MCED tests involves handling a vast amount of complex, heterogeneous data from different sources, including clinical outcomes, genomic data, patient demographics, lifestyle factors, and environmental exposures. The integration of such data from multiple sources and formats (e.g., electronic health records, laboratory results, patient surveys) into a unified database is a technical challenge as it requires consistent data standards and protocols to ensure accuracy and reliability. Further still, cancer biology is highly dynamic, and new discoveries are constantly being made about cancer detection, progression, and treatment, and validation trials must continuously update their protocols and methodologies to reflect new scientific findings and technological advancements, thus adding time and complexity to studies.

The described embodiments represent an improvement to these existing technologies and accordingly address these described technical challenges. In particular, the systems and methods incorporate a data structure that combines data from a prevalence round and one or more incidence rounds, as well as information associated with the detection of interval cancers. This data structure accounts for different demographic factors and clinical characteristics (e.g., age, gender, etc.) in creating an accurate snapshot of cancer detection and screening practices. The systems and methods analyze this data structure in combination with results from prior screening tests (e.g., MCED tests), which results in the determination of risk levels associated with individuals developing specific types of cancers and the establishment of timeframes in which such cancers may develop. The detailed MCED test results described herein also account for the demographics and clinical characteristics of the individuals, and maps these to corresponding data in the data structure. These functionalities enable clinicians to tailor individualized treatment or preventative strategies based on the specific risks, timing, or severity of potential cancer development. These benefits exist without having to conduct large-scale, diverse cohort studies and validation trials for MCED tests, the drawbacks of which are discussed herein.

### System Overview

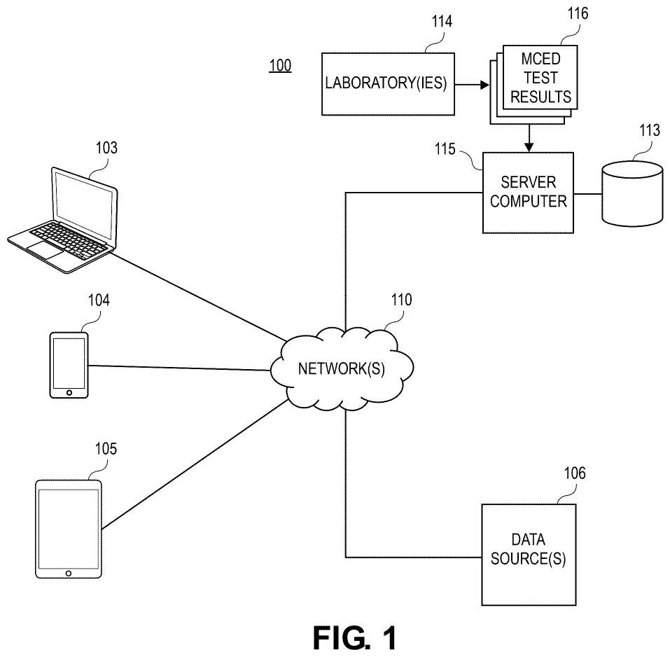

FIG. 1 illustrates an overview of a system 100 of components configured to facilitate the systems and methods. It should be appreciated that the system 100 is merely an example and that alternative or additional components are envisioned.

As illustrated in FIG. 1, the system 100 may include a set of electronic devices 103, 104, 105 which may be used or operated by a set of users, such as any individual or person who may be involved in reviewing results of MCED tests and/or analyses performed on results of MCED tests. For example, the set of users may be an individual who takes an MCED test, a clinician with a patient(s) who takes an MCED test(s), or a combination thereof. Each of the electronic devices 103, 104, 105 may be any type of electronic device such as a mobile device (e.g., a smartphone), desktop computer, notebook computer, tablet, phablet, GPS (Global Positioning System) or GPS-enabled device, smart watch, smart glasses, smart bracelet, wearable electronic, PDA (personal digital assistant), pager, computing device configured for wireless communication, and/or the like.

The electronic devices 103, 104, 105 may communicate with a server computer 115 via one or more networks 110. The server computer 115 may be associated with an entity (e.g., a corporation, company, partnership, or the like) that may be configured to facilitate various of the functionalities as discussed herein. In particular, the server computer 115 may retrieve, obtain, or otherwise access a set of MCED test results 116 related to a set of MCED tests taken by a set of individuals. It should be appreciated that the set of individuals who take the set of MCED tests may or may not overlap with or may or not be the same as the set of individuals associated with the set of electronic devices 103, 104, 105.

Generally, an MCED test is a screening tool designed to detect multiple types of cancers from a single blood sample. Unlike traditional cancer screening methods that target specific cancers (e.g., mammograms for breast cancer or colonoscopies for colorectal cancer), MCED tests aim to detect various cancers early, often before symptoms appear, using advanced genomic and bioinformatics technologies. Generally, MCED tests are designed with a focus on minimizing false positives and false negatives.

An individual who takes an MCED test has a blood sample drawn. A laboratory(ies) 114 may analyze the blood sample to detect and analyze cell-free DNA (cfDNA) circulating in the blood. In particular, cancer cells may release small fragments of DNA into the bloodstream, which are different from normal cell DNA. The laboratory(ies) 114 may employ molecular diagnostic techniques, for example, sequencing and/or PCR to obtain bioinformatic information to examine these DNA fragments for indications of the presence of cancer, for example, specific methylation patterns indicative of cancer.

The result of an MCED test may include an indication of whether a cancer signal is detected or not detected in the blood sample. If a signal is detected, it suggests that there may be cancer present. The result may additionally include a CSO. When a cancer signal is detected in a blood sample, the CSO provides information about the tissue or organ from which the cancer signal likely originates. A result of “no cancer signal detected” may mean that the test did not find any signs of cancer at the time of testing, however it may not guarantee that an individual is cancer-free, as some cancers might not release detectable amounts of DNA into the bloodstream, or the test may not be sensitive enough for all cancer types at very early stages.

A given MCED test may have associated certain demographic factors and/or clinical characteristics of the individual who took the MCED test. In particular, the test result may associate one or more of age, gender, ethnicity and race, family history and genetics (e.g., a family history of cancer or known genetic mutations), lifestyle factors (e.g., smoking status, diet, physical activity, alcohol consumption, environmental exposures, etc.), medical history (e.g., personal history of cancer, previous treatments like chemotherapy or radiation, other health conditions such as chronic infections and autoimmune diseases, etc.), an indication of whether the individual received any prior negative or positive reports/diagnoses, and any other relevant information.

The server computer 115 may support a software application executable by the set of electronic devices 103, 104, 105 (i.e., the set of electronic devices 103, 104, 105 may interface with the server compute 115 in executing the software application), where the user may use the software application to review or communicate information related to MCED test results and analyses thereof. Additionally, the users of the electronic devices 103, 104, 105 may have an account with a service or application offered by the server computer 115. In embodiments, the network(s) 110 may support any type of data communication via any standard or technology (e.g., GSM, CDMA, TDMA, WCDMA, LTE, EDGE, OFDM, GPRS, EV-DO, UWB, Internet, IEEE 802 including Ethernet, WiMAX, Wi-Fi, Bluetooth, and others).

The server computer 115 may be configured to interface with or support a memory or storage 113 capable of storing various data, such as in one or more databases or other forms of storage. According to embodiments, the storage 113 may store data or information associated with MCED test results, analyses of MCED test results, data structures reflecting population-level cancer-related information, reports generated based on the analyses of MCED test results, and/or other data.

The server computer 115 may communicate with one or more data sources 106 via the network(s) 110. In embodiments, the data source(s) 106 may be associated with any organization involved in cancer research. For example, one of the data source(s) 106 may be the American Cancer Society (ACS) Biobank, which is a large-scale research initiative designed to collect and store biological samples, along with detailed health and lifestyle information, from a diverse group of participants, with the goal to support research aimed at understanding cancer development, identifying risk factors, and discovering new methods for cancer prevention, diagnosis, and treatment. For further example, the data source(s) 106 may be the National Cancer Institute (NCI) in the United States which administers Surveillance, Epidemiology, and End Results (SEER) for cancer incidence by age and smoking risk factors. Additionally or alternatively, the data source(s) 106 may store data associated with test sensitivity from the third sub-study of the Circulating Cancer Genome Atlas, and/or duration of detectable window from the American Cancer Society Cancer Prevention Study III. It should be appreciated that alternative and additional data sources are envisioned.

According to embodiments, the server computer 115 may retrieve or otherwise access relevant cancer-related data from the data source(s) 106. In particular, the cancer-related data may include data indicating certain biological samples, health and lifestyle data (e.g., medical histories, including information on prior cancer diagnoses, treatment histories, and other chronic conditions), behavioral data (e.g., lifestyle factors such as diet, exercise habits, smoking status, alcohol consumption, and exposure to environmental risks), demographic information (e.g., age, gender, race/ethnicity, and geographic location), and longitudinal follow-up data to enable the tracking of new cancer diagnoses, changes in health, and outcomes.

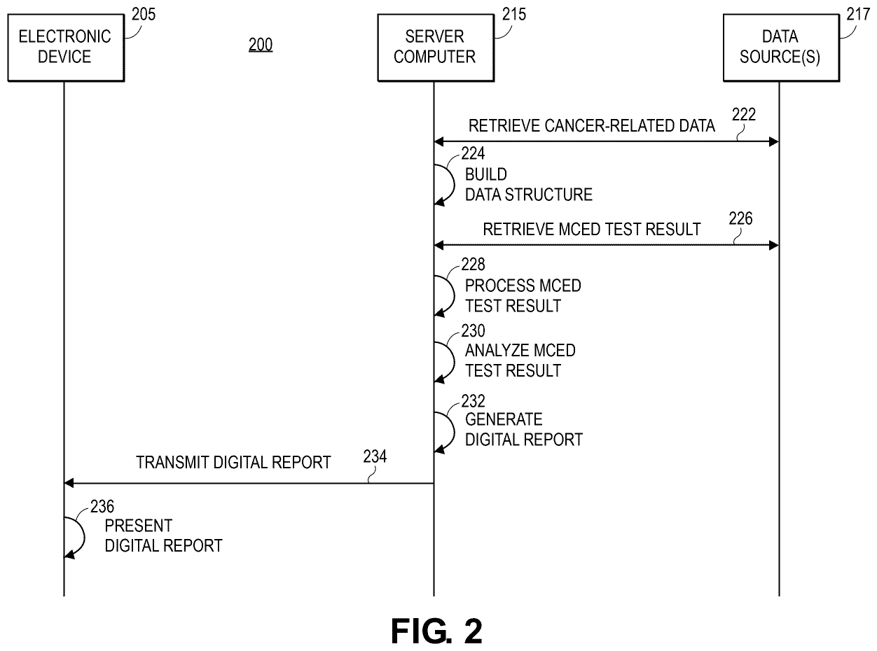

The server computer 115 may review or analyze the cancer-related data, and/or other data, to build or generate a data structure that indicates prevalence round information, incidence round(s) information, and interval cancer information. Further, the server computer 115 may analyze a given MCED test result of an individual from the MCED test results 116 in combination with the data structure to generate a digital report for the individual, where the digital report may include a comprehensive assessment of that MCED test result as well as information associated with a recommended course(s) of action. The server computer 115 may transmit this digital report to one of the electronic devices 103, 104, 105 (or other electronic device) associated with the individual, such as to enable review by the individual and/or a clinician associated with the individual. The server computer 115 may store the digital report and other data associated with these techniques in the storage 113. Additional details regarding these functionalities are further discussed with respect to FIG. 2.

Although depicted as a single server computer 115 in FIG. 1, it should be appreciated that the server computer 115 may be in the form of a distributed cluster of computers, servers, machines, cloud-based services, or the like. In this implementation, the distributed server computer(s) 115 may be utilized as part of an on-demand cloud computing platform. Accordingly, when the electronic devices 103, 104, 105 interface with the server computer 115, the electronic devices 103, 104, 105 may actually interface with one or more of a number of distributed computers, servers, machines, or the like, to facilitate the described functionalities.

Further, although three (3) electronic devices 103, 104, 105 are depicted in FIG. 1, it should be appreciated that greater or fewer amounts are envisioned.

FIG. 2 depicts a signal diagram 200 including various functionalities associated with the described embodiments. The signal diagram 200 may include an electronic device 205 (such as one of the electronic devices 103, 104, 105 as described with respect to FIG. 1), a server computer 215 (such as the server computer 115 as described with respect to FIG. 1), and a data source(s) 217 (such as the data source(s) 106 as described with respect to FIG. 1, or another data source). Although a single electronic device is depicted in and described with respect to FIG. 2, it should be appreciated that multiple electronic devices that may each interface with the server computer 215 are envisioned.

The signal diagram 200 may begin at 222 in which the server computer 215 retrieves cancer-related data from the data source(s) 217. Generally, the cancer-related data may be cancer detection metrics and/or screening outcome data that may encompass certain information on the rates and patterns of cancer detection across different phases of screening within a population, including the initial identification of existing cancers (prevalence), the emergence of new cases between screenings (incidence), and the cancers detected outside of regular screening intervals (interval cancers). In embodiments, the server computer 215 may already store or otherwise have access to the cancer-related data, such as via one or more local or distributed databases.

The server computer 215 may generate or build (224) a data model or data structure using the cancer-related data retrieved in (222). In embodiments, the server computer 215 may access or obtain the data structure that is already generated. Generally, this data structure may track cancer detection rates across different stages of testing and cancer development, and may account for a prevalence round, an incidence round(s), and/or a set of interval cancers. The server computer 215 may perform the calculations and analysis associated with the data of the data structure, as described herein. According to embodiments, the data structure may use an exponential distribution that may model the time between events in a process where events happen continuously and independently at a constant average rate.

Generally, a prevalence round is the first or initial cancer screening test in a population that aims to detect existing, undiagnosed cancers in the population at the time of screening. Any cancers detected in a prevalent screen may be referred to as “prevalent cancers” and may have been present for some time but were previously undetected.

For any individual in the population who participates in the prevalence round and/or incidence round testing, the data structure may associate one or more demographic factors and/or clinical characteristics for that individual. In particular, the demographic factors and/or clinical characteristics may include one or more of age, gender, ethnicity and race, family history and genetics (e.g., a family history of cancer or known genetic mutations), lifestyle factors (e.g., smoking status, diet, physical activity, alcohol consumption, environmental exposures, etc.), medical history (e.g., personal history of cancer, previous treatments like chemotherapy or radiation, other health conditions such as chronic infections and autoimmune diseases, etc.), an indication of whether the individual received any prior negative or positive reports/diagnoses, and any other relevant information.

According to embodiments, the prevalence round may have a Prevalence True Positives (PTP) which refers to the proportion of individuals who test positive for cancer and actually have cancer out of the total population screened during the prevalence round. In an exponential detection window, the PTP may have the equation:

PTP=incidence/year*(test sensitivity)*dwell

Generally, it should be appreciated that the equations identified and described herein may be stratified by the stage at which clinical diagnosis of a cancer(s) would occur if no intervention happened. The incidence/year may refer to the rate at which new cases of a specific cancer are typically diagnosed in a population (e.g., 160 cases per 100,000 individuals) over the course of a year. Further, the test sensitivity refers to the ability of a screening test to correctly identify individuals who actually have cancer, and is calculated as the proportion of true positives (people who have cancer and test positive) out of all individuals who actually have the disease (both true positives and false negatives). Generally, high sensitivity ensures that most cancers present in the population at the time of screening are detected, and low sensitivity results in more false negatives, where people who have cancer are missed by the screening test, leading to underreporting of the PTP.

It should be appreciated that various types of screening tests are envisioned, such as MCED, low-dose computed tomography (LDCT), mammography, colonoscopy, fecal immunochemical test (FIT), stool-based testing, and/or other types of screening tests.

Additionally, the dwell refers to the period during which a cancer is present but remains asymptomatic and undetected (i.e., the time between when a cancer could first be detected (through screening or other tests) and when it becomes clinically evident due to symptoms. Generally, different cancers may have different dwell times, where longer dwell times imply a greater chance to catch the cancer early through regular screening, whereas shorter dwell times suggest that a cancer progresses quickly and might only be detected during an interval between screening rounds. In embodiments, an exponential distribution may be used for modeling cancer dwell time, and the dwell times for different cancers may be fitted to data from the ACS biobank, or to other data.

Different screening tests have varying detection windows, which refer to the time period during which a test can reliably identify cancer before symptoms appear. These detection windows depend on the specific screening method and its sensitivity to the biological markers or physical changes associated with a particular cancer. For example, tests like mammograms and colonoscopies have relatively wide detection windows, allowing them to identify slow-growing cancers or precancerous conditions with long dwell times. In contrast, tests like FITs and stool-based DNA tests have shorter detection windows, as they rely on the presence of specific markers that may only be present during certain stages. The effectiveness of a screening test may depend on its alignment with the dwell time of the cancer being screened for. Cancers with long dwell times, such as many cases of breast or colorectal cancer, are more likely to be detected within routine screening intervals. However, aggressive cancers with short dwell times, such as pancreatic or small-cell lung cancer, may progress too quickly to be detected during the available detection window.

After the prevalence round is performed, subsequent screenings are part of a series of incidence rounds, which focus on detecting newly developed cancers that were not present during the previous screening(s). Typically, the detection rate in an incidence round is lower than in the prevalence round, as the incidence round only captures cancers that have developed in the interval between screenings.

According to embodiments, the incidence round may have an Incidence True Positives (ITP) which refers to the number of new cancer cases that are correctly identified as positive by a screening test during the incidence round, and the ITP rate can help measure the effectiveness of a screening program in detecting new cases that emerge over time, and it can be used to adjust the screening interval to ensure that most cancers are caught as early as possible. The ITP may have the equation:

ITP=PTP*(1−exp(−interval/dwell))

In equation (2), the PTP is calculated using equation (1). Further, the “interval” refers to the time interval between successive screening rounds during which new cancers may develop in individuals who were previously cancer-free at the time of the last screening.

Generally, the longer the interval, the higher the chance that new cancers will develop, and consequently, the more true positive cases might be detected in the next incidence round.

Accordingly, if the interval is too long, some cancers that develop in the interim might progress to later stages or become symptomatic before the next screening round. These cases, which are referred to herein as “interval cancers,” may or may not be detected as true positives during the subsequent incidence screening, depending on whether the cancer remains detectable by the screening method in use. The length of the interval can affect the number of cancers that are detected early (i.e., true positives) vs. those that are missed or diagnosed later (i.e., false negatives). Shorter intervals can improve the ITP rate, as they reduce the window during which cancers can develop undetected.

An Interval False Negative (IFN) refers to a cancer case that develops and becomes symptomatic during the interval between two screenings but is missed or not detected during the prior screening round. In other words, an IFN is a cancer that was present during the earlier screening but was not identified due to limitations in the screening test, and is subsequently diagnosed through other means (e.g., clinical symptoms). In an exponential detection window, the IFN may have the equation:

IFN=Incidence/year*interval−ITP (3)

An IFN typically manifests clinically (i.e., through symptoms) before the next scheduled screening round, highlighting that the cancer was missed despite it being detectable.

Further, an IFN may result from limitations in test sensitivity or aggressive cancer progression. In some cases, the cancer might have been too small or biologically elusive at the time of screening but grows rapidly afterward. A high rate of IFNs can indicate that the screening test may not be sensitive enough, or that the interval between screenings is too long. Monitoring IFN rates helps evaluate the effectiveness of a screening program and can lead to adjustments in the frequency or type of screening performed. It should be appreciated that the time interval from the prior screening round may be known.

According to embodiments, the server computer 215 may measure an episode sensitivity, such as in the context of an active intervention (e.g., a screening program, a diagnostic test, or another clinical action). The episode sensitivity may be measured in a prevalence round for one interval according to the following equation:

Episode sensitivity=PTP/(IFN+PTP) (4)

Additionally or alternatively, the episode sensitivity may be measured in an incidence round for one interval according to the following equation:

Episode sensitivity=ITP/(IFN+ITP) (5)

It should be appreciated that the test as included as part of the data structure may be an MCED test, where the data structure may account for a sensitivity of the MCEDs, a false positive rate of the MCEDs in the reference population, expected duration of preclinical detection in the reference population, and an incidence of cancer in the reference population, any CSO signals included in the MCED results, and/or other information.

According to embodiments, the resulting data structure may include or reflect a prevalence dataset that may encompass data from a prevalence round, an incidence dataset that may encompass data from one or more incidence rounds, and/or a third dataset that may encompass data indicating diagnoses of interval cancers.

The server computer 215 may retrieve, access, or obtain (226) a result of an MCED test administered to an individual. In embodiments, a blood sample that is drawn for a given MCED test may be analyzed by a laboratory, which assesses a result of the MCED test. The result may include an indication of whether a cancer signal is detected or not detected in the blood sample. If a signal is detected, the result may additionally include a CSO. Along with the MCED test result, the server computer 215 may also retrieve, access, or obtain information on the individual who took the MCED test, such as age, sex, smoking status, results of any prior MCED tests or screening tests results and the time intervals/dates of such tests, and so on. Although described as the server computer 215 retrieving a single MCED test result for a single individual, it should be appreciated that the server computer 215 may retrieve multiple MCED test results respectively corresponding to multiple individuals.

In embodiments, the server computer 215 may process (228) the result of the MCED test. In particular, the server computer 215 may determine or incorporate relevant demographic information and/or clinical characteristics about the individual who took the test. This processing may involve adjusting the test outcomes and their interpretations based on factors like age, gender, ethnicity, family history, and other personal health characteristics.

The server computer 215 may analyze (228) the result of the MCED test in combination with the data structure that includes the prevalence dataset, the incidence dataset, and/or the third dataset (i.e., indicating diagnoses of interval cancers). In analyzing the result, the server computer 215 may determine a set of risk levels of the individual developing one or more of a plurality of cancers over a specified time period (e.g., six months, a year, or other time periods). According to embodiments, the set of risk levels may account for one or more demographic factors and/or clinical characteristics of the individual. For example, the set of risk levels may account for at least an age and a gender of the individual. It should be appreciated that the set of percentage risks may account for different combinations of different demographic factors and/or clinical characteristics.

To analyze the result of the MCED test in combination with the data structure, the server computer 215 may integrate the data by mapping any CSO included in the MCED result to the corresponding cancer(s) in the data structure, and ensure that the timescales are compatible. In particular, the server computer 215 may align relevant demographics and/or clinical characteristics of the individual (e.g., age, gender, time since last screening, etc.) with corresponding data in the prevalence and/or incidence rounds in the data structure. Generally, the server computer 215 may facilitate a similarity measure technique to match the individual to a reference population as included in the data structure, based on a set of observable clinical values of the individual (e.g., age, gender, smoking status, etc.).

The server computer 215 may use additional or alternative techniques as part of this data analysis. In particular, the server computer 215 may employ risk stratification by using the risk levels of an individual to place the individual into appropriate risk categories for each cancer type, and comparing these risk levels to the overall population risk represented in the data structure; a Bayesian analysis by using the cancer detection rates from the data structure as prior probabilities, and updating these prior probabilities with the risk factors of the individual to calculate personalized posterior probabilities for each cancer type; a time-dependent analysis by considering how the risk of an individual changes over time in relation to the prevalence round, incidence round, and interval cancer data; a multi-cancer consideration analysis by analyzing potential interactions between different cancer risks for the individual, and assessing patterns in the data structure that might indicate correlations between different cancer types; and/or a screening recommendation analysis by determining optimal screening intervals for the individual for each cancer type and comparing these to standard screening guidelines to identify any necessary adjustments. It should be appreciated that alternative or additional types of analyses are envisioned.

It should be appreciated that each risk level of the set of risk levels may be in the form of a percentage likelihood (e.g., 0%-100%), a numerical risk level (e.g., a number from 0-10), a risk category (e.g., low, medium, or high), and/or another indication type.

Based on the results of the analysis of (230), the server computer 215 may generate (232) an electronic or digital document, report, chart, and/or the like (as referred to herein as a “digital report”) that may include relevant information from the analysis of the MCED test result of an individual. Generally, the server computer 215 may determine what types of data or information each digital report should include, for instance based on the MCED test result, one or more demographic factors and/or clinical characteristics of the individual, and/or other factors.

In embodiments, a given digital report may indicate whether a cancer signal was detected in the MCED test result. Further, if a cancer signal was not detected, the digital report may indicate a risk level, of the set of risk levels determined by the server computer 215, of the individual having or developing at least one cancer, of a plurality of cancers, over a specified time period(s). Additionally, if a cancer signal was detected, and for each of at least some of the cancers of the plurality of cancers, an additional risk level, of the set of risk levels, that a screening test corresponding to that cancer would result in the individual being diagnosed with that cancer. In some cases, one or more risk levels associated with precancerous conditions may be indicated. Merely by way of example, such precancerous conditions can include conditions that are recognized as having a significantly increased risk of developing into cancer, such as certain general/multi-organ premalignant conditions (e.g., dysplasia, carcinoma in situ, adenoma/adenomatous polyps, leukoplakia); precancerous conditions in the gastrointestinal tract such as esophagus (e.g., Barrett's esophagus, esophageal squamous dysplasia), stomach (chronic atrophic gastritis, intestinal metaplasia, gastric adenoma, dysplasia of gastric mucosa), colon and rectum (e.g., adenomatous polyps, sessile serrated lesions/traditional serrated adenomas, inflammatory bowel disease-related dysplasia); hepatobiliary precancerous conditions (e.g., cirrhosis, hepatic dysplastic nodules, primary sclerosing cholangitis, biliary intraepithelial neoplasia); pancreas (e.g., pancreatic intraepithelial neoplasia, intraductal papillary mucinous neoplasms, mucinous cystic neoplasms); breast precancerous conditions (e.g., atypical ductal hyperplasia, atypical lobular hyperplasia, ductal carcinoma in situ, lobular carcinoma in situ); gynecological precancerous conditions (e.g., cervix-related such as cervical intraepithelial neoplasia and adenocarcinoma in situ, endometrium-related such as endometrial hyperplasia and endometrial intraepithelial neoplasia, vulva/vagina-related such as vulvar intraepithelial neoplasia, vaginal intraepithelial neoplasia); urologic precancerous conditions (e.g., prostatic intraepithelial neoplasia, urothelial dysplasia, carcinoma in situ of the bladder); skin precancerous conditions (e.g., actinic keratosis, dysplastic nevus, Bowen's disease, erythroplasia of Queyrat); head and neck precancerous conditions (e.g., oral leukoplakia, laryngeal dysplasia); thyroid precancerous conditions (e.g., follicular neoplasm/atypical follicular adenoma, non-invasive follicular thyroid neoplasm); hematologic precancerous conditions (e.g., MDS, MGUS, CHIP); lung precancerous conditions (e.g., atypical adenomatous hyperplasia, squamous dysplasia, DIPNECH).

It should be appreciated that the plurality of cancers may include various types of cancer types with varying degrees of lethality. For example, certain cancers have a high lethality (e.g., pancreatic and lung cancers), moderate lethality (e.g., prostate and colorectal cancers), or varied lethality (e.g., breast cancer and melanoma).

The server computer 215 may transmit (234) the digital report to the electronic device 205, such as via a network connection. The electronic device 205 may be associated with an individual who took the MCED test, or may be a clinician or other healthcare individual who is caring for or otherwise advising the individual. In embodiments, the server computer 215 may avail the digital report for access by the electronic device 205, such as via an application (e.g., a web browser, email, a dedicated application or program, a portal, an electronic health record (EHR) program), and/or the like. Alternatively or additionally, the digital report may be in the form of a digital spreadsheet that may enable adjustment of the underlying data structure. It should be appreciated that the server computer 215 may transmit the digital report to the electronic device 205, where the electronic device 205 may initiate an application that may be configured to render or display the digital reports and content thereof, such as via an interface associated with the application. For example, the application may be a web browser and the interface may be a webpage associated with a website. In embodiments, the individual associated with the electronic device 205 may select to initiate the application, and the application may initiate in response to the selection.

The electronic device 205 may present or render (236) the digital report, such as via a user interface of the electronic device and within an interface of the application. In this regard, an individual may review the digital report to make more informed decisions about interventions, treatments, testing, screening, and/or the like. It should be appreciated that additional benefits are envisioned. In embodiments, the individual associated with the electronic device 205 may select, via the user interface, to review the digital report, and the electronic device 205 may render the digital report in response to receiving this selection.

### Digital Reporting of Cancer Prediction Results

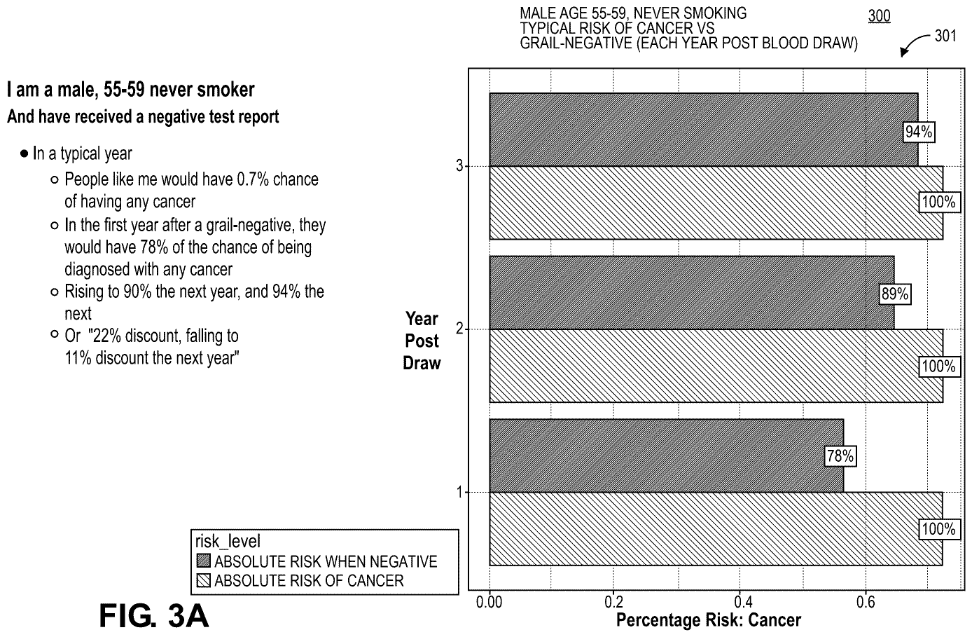

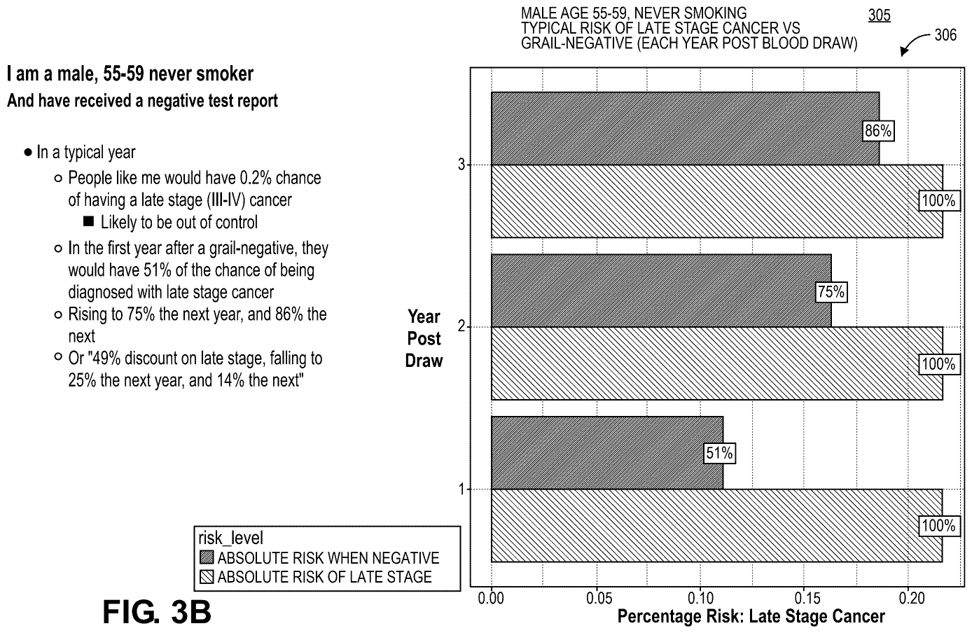

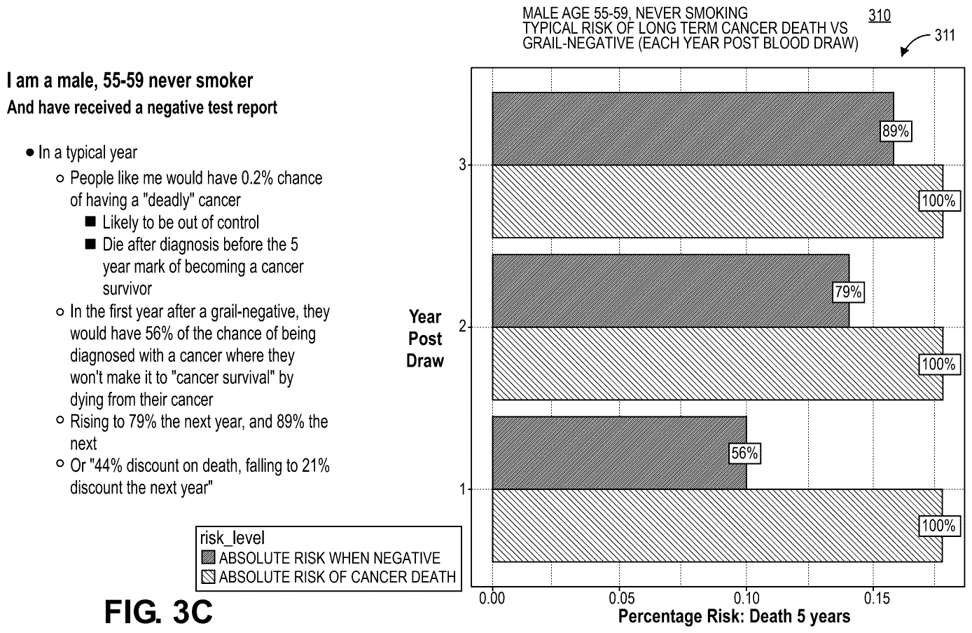

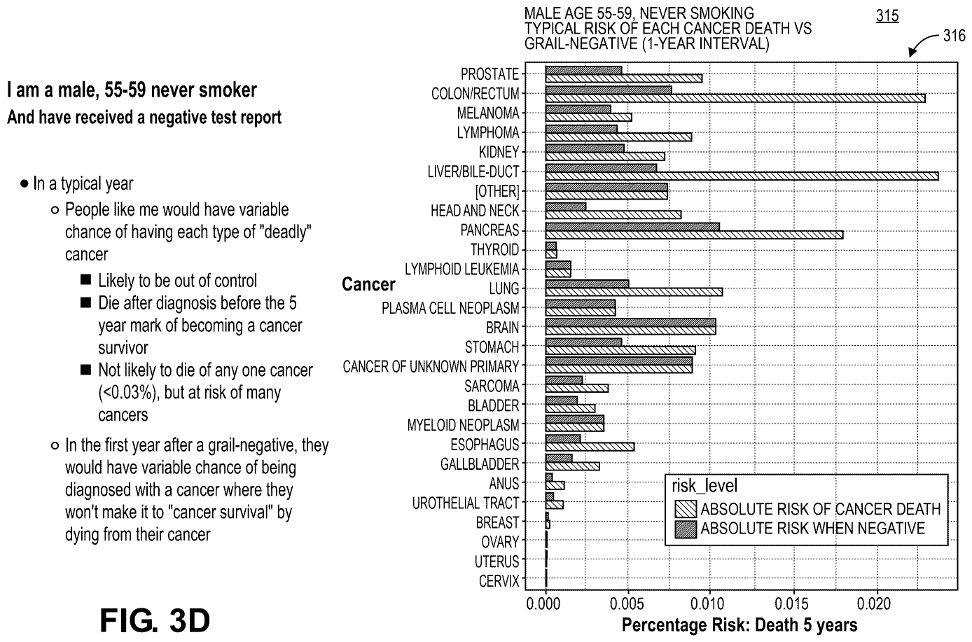

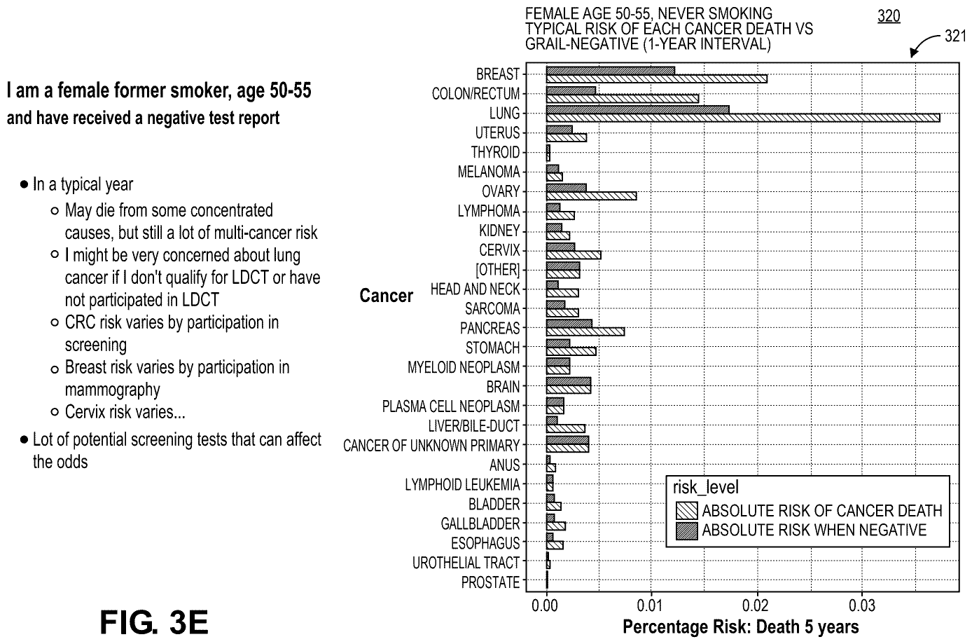

FIG. 3A depicts an example digital report 300 that may be generated by the server computer 215. The digital report 300 indicates that a cancer signal was not detected for the tested individual, and further indicates various risk levels resulting from the analysis of the MCED test result in combination with the data structure. For example, the digital report 300 indicates that a given individual in the general population of males aged 55-59 who never smoke has a 0.7% chance of having any cancer. The digital report 300 further indicates that a given individual who takes the MCED test and whose MCED test result indicates that a cancer signal was not detected has a 78% percent of that 0.7% chance (i.e., 0.546%) of being diagnosed with any cancer in the first year after taking the MCED test. Additionally, the digital report 300 indicates that this risk level increases to 90% of the 0.7% chance (i.e., 0.63%) in the second year after taking the MCED test, and to 95% of the 0.7% chance (i.e., 0.658%) in the third year after taking the MCED test. The digital report 300 may include a chart 301 that illustrates this set of risk levels. For example, the chart may compare risk levels compared to statistics of cancer diagnosis for a stratum of individuals matching the individual's demographics. The report may further indicate: “In a typical year: people like me would have 0.7% chance of having any cancer, in the first year after a Grail-Negative; they would have 78% chance of being diagnosed with any cancer; rising to 90% in the next year, and to 95% in the next year after than; or ‘22%’ discount, falling to 11% discount the next year.’”

FIG. 3B depicts another example digital report 305 that may be generated by the server computer 215. The digital report 305 indicates that a cancer signal was not detected for the tested individual, and further indicates various risk levels resulting from the analysis of the MCED test result in combination with the data structure. For example, the digital report 305 indicates that a given individual in the general population of males aged 55-59 who never smoke has a 0.2% chance of having a late-stage (i.e., stage III or IV) cancer. The digital report 305 further indicates that a given individual who takes the MCED test and whose MCED test result indicates that a cancer signal was not detected has a 51% percent of that 0.2% chance (i.e., 0.0102%) of being diagnosed with a late stage cancer in the first year after taking the MCED test. Additionally, the digital report 305 indicates that this risk level increases to 75% of the 0.2% chance (i.e., 0.015%) in the second year after taking the MCED test, and to 86% of the 0.2% chance (i.e., 0.0172%) in the third year after taking the MCED test. The digital report 305 may include a chart 306 that illustrates this set of risk levels. The chart may compare risk levels compared to statistics of cancer diagnosis for a stratum of individuals matching the individual's demographics. For example, the report may further indicate: “In a typical year: people like me would have 0.2% chance of having a late-stage (III-IV) cancer, likely to be out of control; in the first year after a Grail-Negative, they would have 51% chance of being diagnosed with late-stage cancer; rising to 75% in the next year, and to 86% in the next year after than; or ‘49%’ discount, falling to 25% discount the next year, and to 14% discount in the next year after that.’”

FIG. 3C depicts another example of a digital report 310 that may be generated by the server computer 215. The digital report 310 indicates that a cancer signal was not detected for the tested individual, and further indicates various risk levels resulting from the analysis of the MCED test result in combination with the data structure. For example, the digital report 310 indicates that a given individual in the general population of males aged 55-59 who never smoke has a 0.2% chance of having a “deadly” cancer (e.g., a cancer in a class of cancers that comprise a majority of all cancer deaths, such as anus, bladder, colon/rectum, esophagus, head and neck, liver/bile duct, lung, lymphoma, ovary, pancreas, plasma cell neoplasm, and stomach). Similar to the digital reports 300, 305, the digital report 310 further indicates certain risk levels of being diagnosed with a deadly cancer, across time periods, for a given individual whose MCED test result indicates that a cancer signal was not detected. The digital report 310 may include a chart 311 that illustrates this set of risk levels. The chart may compare risk levels compared to statistics of cancer outcome for a stratum of individuals matching the individual's demographics. For example, the report may further indicate: “In a typical year: people like me would have 0.2% chance of having a ‘deadly’ cancer, likely to be out of control, die after diagnosis before the 5-year mark of becoming a cancer survivor; in the first year after a Grail-Negative, they would have 56% chance of being diagnosed with a cancer where they won't make it to ‘cancer survival’ by dying from their cancer; rising to 79% in the next year, and to 89% in the next year after than; or ‘44%’ discount on death, falling to 21% discount the next year.’”

FIG. 3D depicts another example digital report 315 that may be generated by the server computer 215. The digital report 315 indicates that a cancer signal was not detected for the tested individual, and further indicates various risk levels resulting from the analysis of the MCED test result in combination with the data structure. For example, the digital report 315 indicates various risk levels that a given individual in the general population of males aged 55-59 who never smoke has any of a set of cancers or deadly cancers (e.g., prostate, colon/rectum, melanoma, lymphoma, etc.). Further, the digital report 315 indicates how these risk levels are adjusted for a given male whose MCED test result indicates that a cancer signal was not detected. The digital report 315 may include a chart 316 that illustrates this set of risk levels. The chart 316 generally indicates that the risk of a given male having certain cancers (e.g., prostate, colon/rectum, pancreas) is reduced after a negative MCED test result, and that the risk of a given male having other cancers (e.g., plasma cell neoplasm, brain, and CUP) is unchanged after a negative MCED test result. The chart may compare risk levels compared to statistics of cancer outcome for a stratum of individuals matching the individual's demographics. For example, the report may further indicate: “In a typical year: people like me would have variable chance of having each type of ‘deadly’ cancer, likely to be out of control, die after diagnosis before the 5-year mark of becoming a cancer survivor, not likely to die of any one cancer (<0.03%), but at risk of many cancers; in the first year after a Grail-Negative, they would have a variable chance of being diagnosed with a cancer where they won't make it to ‘cancer survival’ by dying from their cancer.”

FIG. 3E depicts another example digital report 320 that may be generated by the server computer 215. The digital report 320 is similar to the digital report 315 of FIG. 3D, and indicates that a cancer signal was not detected for the tested individual, and further indicates various risk levels resulting from the analysis of the MCED test result in combination with the data structure. For example, the digital report 325 indicates various risk levels that a given individual in the general population of females aged 55-59 who never smoke has any of a set of cancers (e.g., breast, colon/rectum, lung, uterus, etc.), such as any of a set of cancers included in the individual's MCED test. Further, the digital report 320 indicates how these risk levels are adjusted for a given female whose MCED test result indicates that a cancer signal was not detected. The digital report 320 may include a chart 321 that illustrates this set of risk levels within a period of time (e.g., 5 years). The chart 321 generally indicates that the risk of a given female having certain cancers (e.g., breast, colon/rectum, lung) is reduced after a negative MCED test result, and that the risk of a given female having other cancers (e.g., plasma cell neoplasm, brain, and CUP) is unchanged after a negative MCED test result. The chart may compare risk levels compared to statistics of cancer outcome for a stratum of individuals matching the individual's demographics. For example, the report may further indicate: “In a typical year: may die from some concentrated causes, but still a lot of multi-cancer risk; I might be very concerned about lung cancer if I don't qualify for LDCT or have not participated in LDCT; CRC people like me would have variable chance of having each type of ‘deadly’ cancer, likely to be out of control, die after diagnosis before the 5-year mark of becoming a cancer survivor, not likely to die of any one cancer (<0.03%), but at risk of many cancers; in the first year after a Grail-Negative, they would have a variable chance of being diagnosed with a cancer where they won't make it to ‘cancer survival’ by dying from their cancer.”

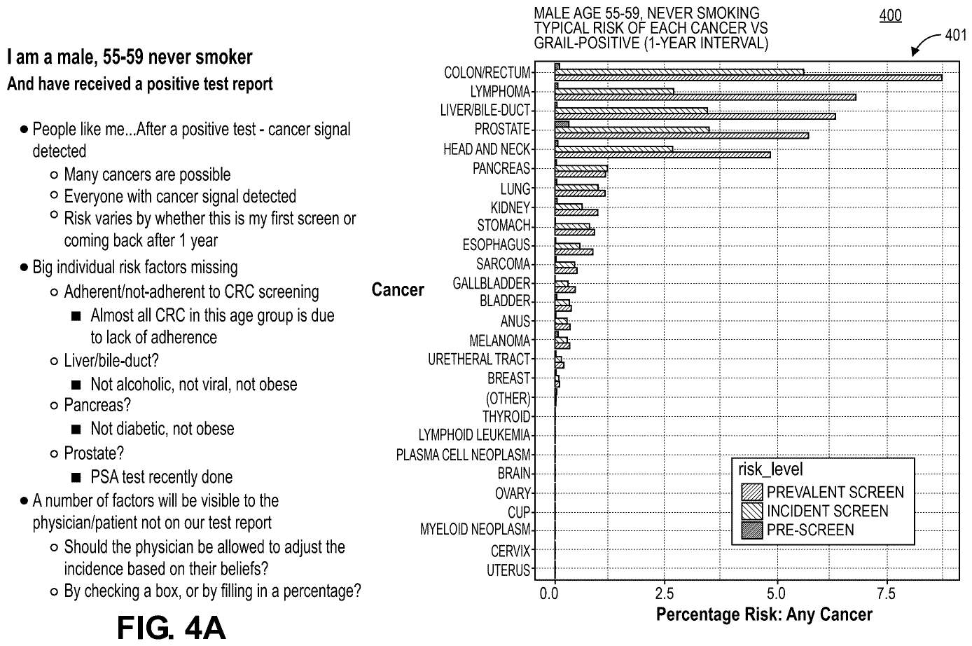

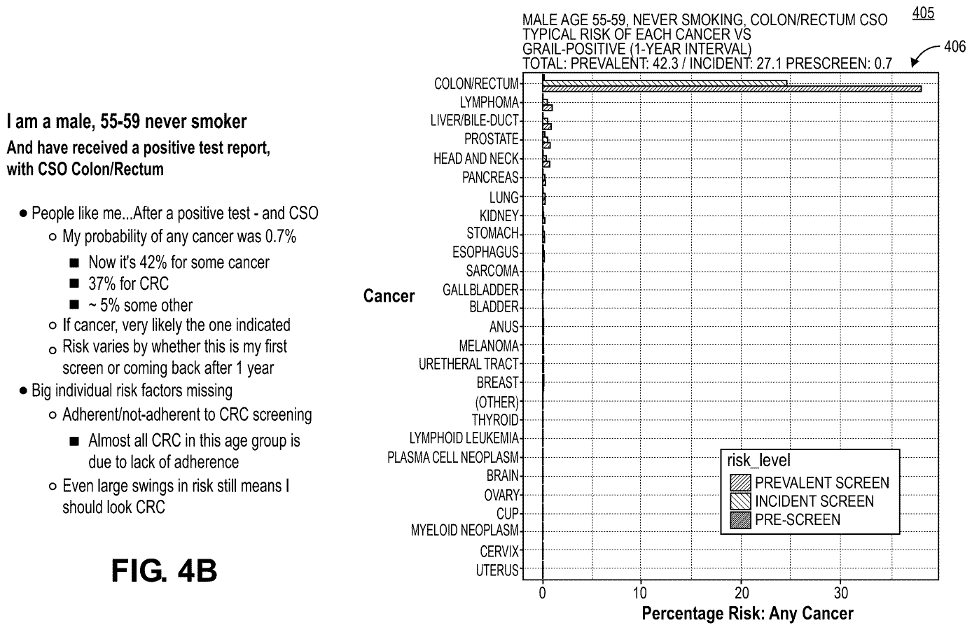

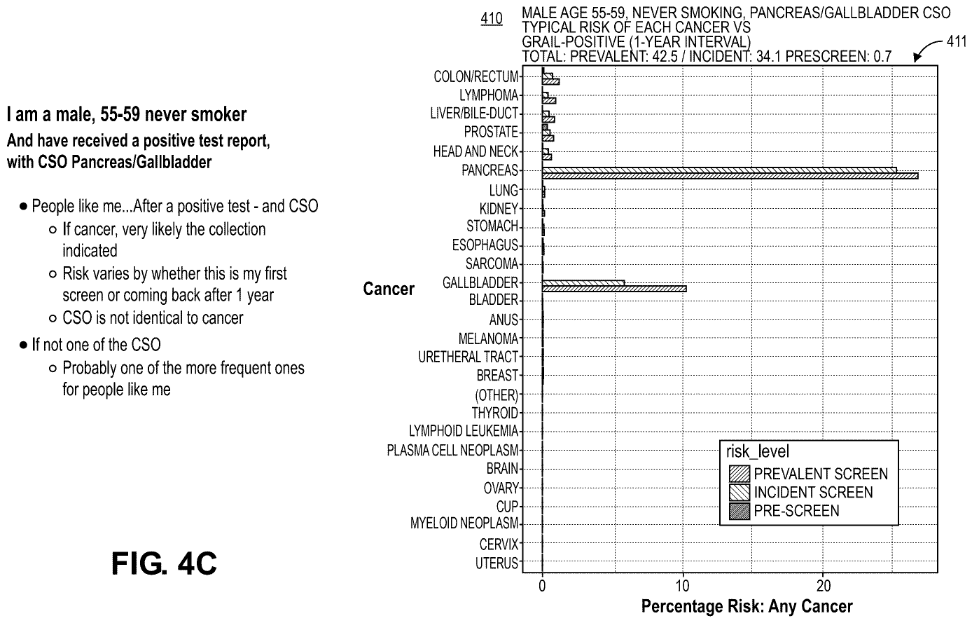

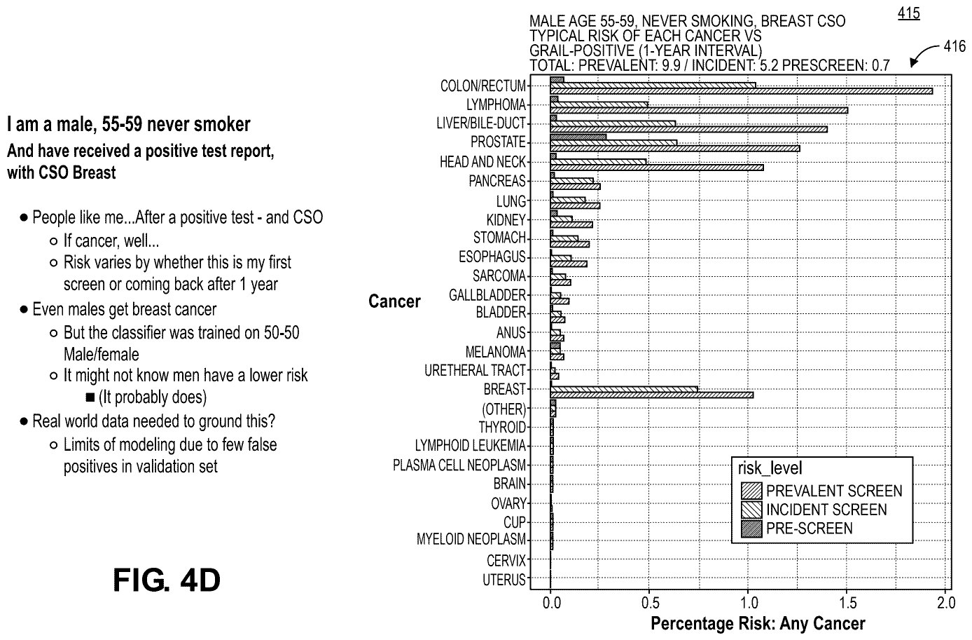

FIG. 4A depicts an example digital report 400 that may be generated by the server computer 215. The digital report 400 indicates that a cancer signal was detected for the tested individual, and further indicates various risk levels resulting from the analysis of the MCED test result in combination with the data structure. The server computer 215 may generate the digital report 400 in the event that, for example, the MCED test does not indicate a CSO signal.

The digital report 400 may include a chart 401 that illustrates various of these risk levels across a plurality of cancers (e.g., colon/rectum, lymphoma, liver/bile-duct, etc.). In particular, the chart 401 indicates, for each cancer of the plurality of cancers, (i) a pre-screen risk level, (ii) an incident screen risk level, and (iii) a prevalent screen risk level. Each pre-screen risk level may be the risk of a given individual who does not undertake a screening test being diagnosed with that specific cancer in the next year; each prevalent screen risk level may be the risk of a given individual having a positive MCED test result being diagnosed with that specific cancer in the next year, if this is the first MCED test result for that given individual; and each incident screen risk level may be the risk of a given individual having a positive MCED test result being diagnosed with that specific cancer in the next year, if this is the second or other subsequent MCED test result for that given individual.

FIG. 4B depicts an example digital report 405 that may be generated by the server computer 215. The digital report 405 indicates that a cancer signal was detected for the tested individual, a CSO for the cancer signal (colon/rectum), and further indicates various risk levels resulting from the analysis of the MCED test result in combination with the data structure.

The digital report 405 may include a chart 406 that illustrates various of these risk levels across a plurality of cancers (e.g., colon/rectum, lymphoma, liver/bile-duct, etc.). In particular, the chart 406 indicates, for each cancer of the plurality of cancers, (i) a pre-screen risk level, (ii) an incident screen risk level, and (iii) a prevalent screen risk level, as described herein. Because the digital report 405 indicates a CSO of colon/rectum cancer, the incident screen and prevalent screen risk levels for this particular cancer are significantly higher than those of the other cancers. However, just because the MCED result of a given individual has a CSO for a particular cancer does not mean that that individual will develop or be diagnosed with that cancer. Accordingly, the digital report 405 may enable the individual and any clinician to make more informed decisions about interventions, treatments, and/or the like.

FIG. 4C depicts an example digital report 410 that may be generated by the server computer 215. The digital report 410 indicates that a cancer signal was detected for the tested individual, a CSO for the cancer signal (pancreas/gallbladder), and further indicates various risk levels resulting from the analysis of the MCED test result in combination with the data structure.

The digital report 410 may include a chart 411 that illustrates various of these risk levels across a plurality of cancers (e.g., colon/rectum, lymphoma, liver/bile-duct, etc.). In particular, the chart 411 indicates, for each cancer of the plurality of cancers, (i) a pre-screen risk level, (ii) an incident screen risk level, and (iii) a prevalent screen risk level, as described herein. Because the digital report 410 indicates a CSO of pancreas/gallbladder cancer, the incident screen and prevalent screen risk levels for those particular cancers are significantly higher than those of the other cancers.

FIG. 4D depicts an example digital report 415 that may be generated by the server computer 215. The digital report 415 indicates that a cancer signal was detected for the tested male individual, a CSO for the cancer signal (breast), and further indicates various risk levels resulting from the analysis of the MCED test result in combination with the data structure.

The digital report 415 may include a chart 416 that illustrates various of these risk levels across a plurality of cancers (e.g., colon/rectum, lymphoma, liver/bile-duct, breast, etc.).

In particular, the chart 416 indicates, for each cancer of the plurality of cancers, (i) a pre-screen risk level, (ii) an incident screen risk level, and (iii) a prevalent screen risk level, as described herein. Because the digital report 416 indicates a CSO of breast, the incident screen and prevalent screen risk levels for this particular cancer are elevated. However, because breast cancer in males is somewhat rare, the incident screen and prevalent screen risk levels for other listed cancers (e.g., colon/rectum, lymphoma, liver/bile-duct) are also elevated. That is, even though the digital report 416 indicates a CSO of breast, the particular individual is at an elevated risk of being diagnosed with cancers other than breast cancer.

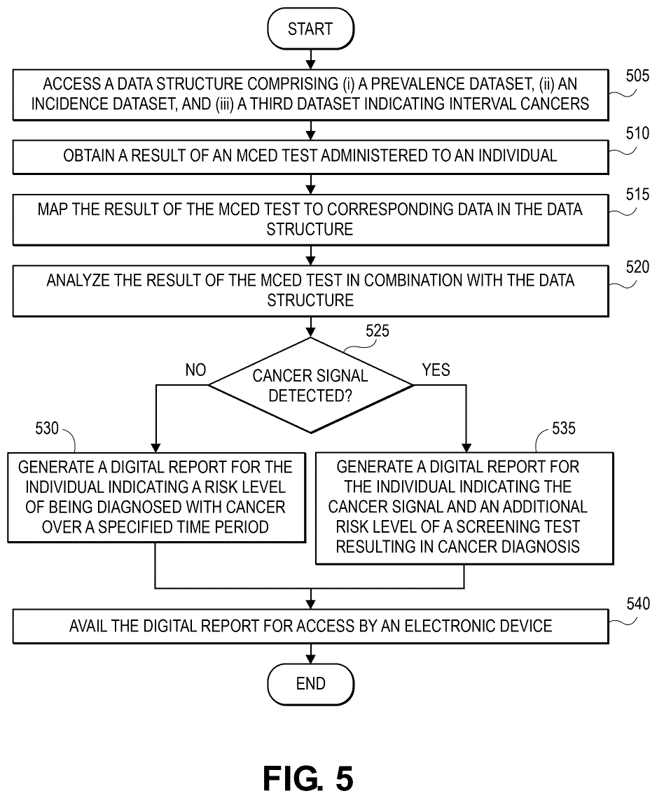

FIG. 5 is a block diagram of an example method 500 of ascertaining cancer-related risks resulting from an MCED test administered to an individual using population-level classification data. The method 500 may be facilitated by one or more electronic devices (such as the server computer 115 as described with respect to FIG. 1).

The method 500 may begin when the electronic device accesses (block 505) a data structure that may include (i) a prevalence dataset indicating an initial prevalence of a plurality of individuals who have one or more of a plurality of cancers, (ii) an incidence dataset indicating an incidence of the plurality of individuals who are newly diagnosed with one or more of the plurality of cancers within a specified time period, (iii) a third dataset indicating instances of the plurality of individuals being diagnosed with one or more of the plurality of cancers between screening tests, or some combination thereof.

The electronic device may obtain (block 510) a result of the MCED test administered to the individual. In embodiments, the result of the MCED test may indicate whether a cancer signal was detected. In embodiments, if the result of the multi-cancer early detection (MCED) test indicates that the cancer signal was detected, the result of the MCED test further may further indicate a cancer signal origin (CSO) for the cancer signal.

The electronic device may map (block 515) the result of the MCED test to the corresponding data structure. In embodiments, the electronic device may map an age and gender of the individual to corresponding data within the data structure, resulting in a set of mapped data. It should be appreciated that the electronic device may map alternative or additional demographic factors and/or clinical characteristics.

The electronic device may analyze (block 520) the result of the MCED test in combination with the data structure (e.g., in the form of the set of mapped data) to determine a set of risk levels of the individual developing one or more of the plurality of cancers over the specified time period, wherein the set of risk levels accounts for at least the age and the gender of the individual. In embodiments, each risk level in the set of risk levels may be a percentage likelihood, a category, or another type of risk indication.

The electronic device may determine (block 525) whether the result of the MCED test indicates the detection of a cancer signal. If the cancer signal is not detected (“NO”), processing may proceed to block 530 in which the electronic device may generate a digital report for the individual, where the digital report may indicate that the cancer signal was not detected, and a risk level, of the set of risk levels, of the individual being diagnosed with at least one cancer, of the plurality of cancers, over the specified time period. Processing may then proceed to block 540.

If the cancer signal is detected (“YES”), processing may proceed to block 535 in which the electronic device may generate a digital report for the individual, where the digital report may indicate the cancer signal and, for each of at least some of the cancers of the plurality of cancers, an additional risk level, of the set of risk levels, that a screening test corresponding to that cancer would result in the individual being diagnosed with that cancer. Processing may then proceed to block 540.

According to embodiments, the digital report may include alternative or additional information, which may be based on whether the cancer signal was detected. For instance, the digital report may indicate (i) that the cancer signal was not detected, (ii) a general risk level of another individual of the age and the gender of the individual having cancer, (iii) an individual risk level of the individual being diagnosed with the at least one cancer, of the plurality of cancers, over the specified time period.

In another situation, the digital report may indicate (i) that the cancer signal was not detected, (ii) a plurality of general risk levels of another individual of the age and the gender of the individual respectively having the plurality of cancers, (iii) a plurality of individual risk levels of the individual respectively being diagnosed with the plurality of cancers over the specified time period.

In a further situation, the digital report may indicate (i) that the cancer signal was detected, (ii) for each of the plurality of cancers, a pre-screen risk level of another individual of the age and the gender of the individual being diagnosed with that cancer absent screening, (iii) for each of the plurality of cancers, a prevalent screen risk level of the individual being diagnosed with that cancer over the specified time period, and (iv) for each of the plurality of cancers, an incident screen risk level of the individual being diagnosed with that cancer over another time period subsequent to the specified time period.

In a still further situation, the digital report may indicate (i) that the cancer signal was detected, (ii) a cancer signal origin (CSO) for the cancer signal, (iii) for each of the plurality of cancers, a pre-screen risk level of another individual of the age and the gender of the individual being diagnosed with that cancer absent screening, (iv) for each of the plurality of cancers, a prevalent screen risk level of the individual being diagnosed with that cancer over the specified time period, and (v) for each of the plurality of cancers, an incident screen risk level of the individual being diagnosed with that cancer over another time period subsequent to the specified time period.

The electronic device may avail (block 540) the digital report for access by an electronic device. In embodiments, the electronic device may be associated with the individual who initially took the MCED test, a clinician who is treating the individual, or another user.

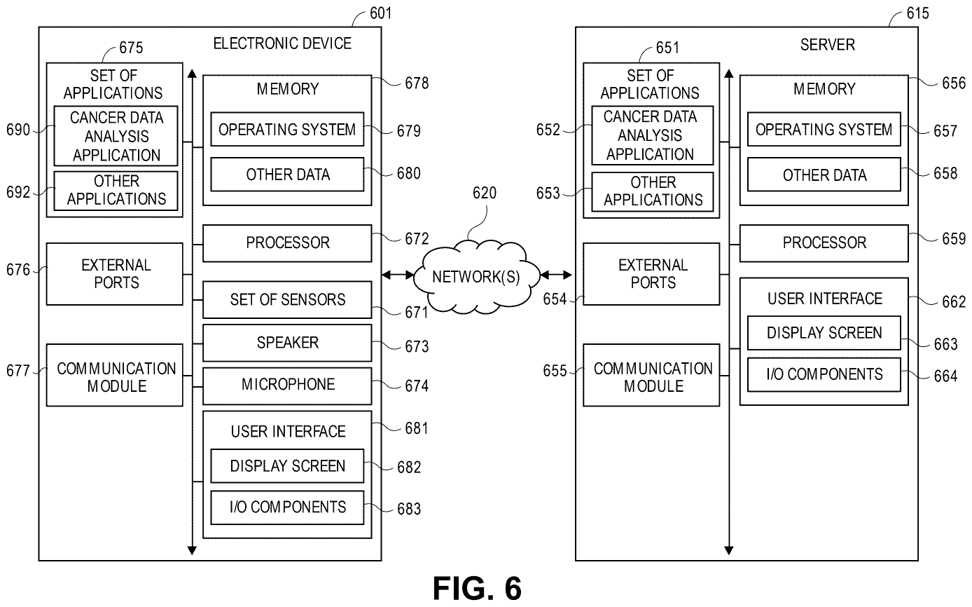

FIG. 6 illustrates a hardware diagram of an example electronic device 601 (e.g., one of the electronic devices 103, 104, 105 as described with respect to FIG. 1A) and an example server 615 (e.g., the server computer 115 as described with respect to FIG. 1A), in which the functionalities as discussed herein may be implemented. It should be appreciated that the components of the electronic device 601 and the server 615 are merely exemplary, and that additional or alternative components and arrangements thereof are envisioned.

The electronic device 601 may include a processor 672 as well as a memory 678. The memory 678 may store an operating system 679 capable of facilitating the functionalities as discussed herein as well as a set of applications 675 (i.e., machine readable instructions). For example, one of the set of applications 675 may be a cancer data analysis application 690, such as to access various cancer-related data, build or generate data structures, analyze MCED test results in combination with the data structures, generate and/or present digital reports associated with the analyses, and/or other functionalities. It should be appreciated that one or more other applications 692 are envisioned.

The processor 672 may interface with the memory 678 to execute the operating system 679 and the set of applications 675. According to some embodiments, the memory 678 may also store other data 680, such as cancer-related data that may be used in the analyses and determinations as discussed herein. The memory 678 may include one or more forms of volatile and/or non-volatile, fixed and/or removable memory, such as read-only memory (ROM), electronic programmable read-only memory (EPROM), random access memory (RAM), erasable electronic programmable read-only memory (EEPROM), and/or other hard drives, flash memory, MicroSD cards, and others.

The electronic device 601 may further include a communication module 677 configured to communicate data via one or more networks 610. According to some embodiments, the communication module 677 may include one or more transceivers (e.g., WAN, WWAN, WLAN, and/or WPAN transceivers) functioning in accordance with IEEE standards, 3GPP standards, or other standards, and configured to receive and transmit data via one or more external ports 676.

The electronic device 601 may include a set of sensors 671 such as, for example, a location module (e.g., a GPS chip), an image sensor, an accelerometer, a clock, a gyroscope (i.e., an angular rate sensor), a compass, a yaw rate sensor, a tilt sensor, telematics sensors, and/or other sensors. The electronic device 601 may further include a user interface 681 configured to present information to a user and/or receive inputs from the user. As shown in FIG. 6, the user interface 681 may include a display screen 682 and I/O components 683 (e.g., ports, capacitive or resistive touch sensitive input panels, keys, buttons, lights, LEDs, and/or built in or external keyboard). Additionally, the electronic device 601 may include a speaker 673 configured to output audio data and a microphone 674 configured to detect audio.

In some embodiments, the electronic device 601 may perform the functionalities as discussed herein as part of a “cloud” network or may otherwise communicate with other hardware or software components within the cloud to send, retrieve, or otherwise analyze data.

As illustrated in FIG. 6, the electronic device 601 may communicate and interface with the server 615 via the network(s) 610. The server 615 may include a processor 659 as well as a memory 656. The memory 656 may store an operating system 657 capable of facilitating the functionalities as discussed herein as well as a set of applications 651 (i.e., machine readable instructions). For example, one of the set of applications 651 may be a cancer data analysis application 652, such as to access various cancer-related data, build or generate data structures, analyze MCED test results in combination with the data structures, generate and/or present digital reports associated with the analyses, and/or other functionalities. It should be appreciated that one or more other applications 653 are envisioned.

The processor 659 may interface with the memory 656 to execute the operating system 657 and the set of applications 651. According to some embodiments, the memory 656 may also store other data 658, such as cancer-related data that may be used in the analyses and determinations as discussed herein. The memory 656 may include one or more forms of volatile and/or nonvolatile, fixed and/or removable memory, such as read-only memory (ROM), electronic programmable read-only memory (EPROM), random access memory (RAM), erasable electronic programmable read-only memory (EEPROM), and/or other hard drives, flash memory, MicroSD cards, and others.

The server 615 may further include a communication module 655 configured to communicate data via the one or more networks 610. According to some embodiments, the communication module 655 may include one or more transceivers (e.g., WAN, WWAN, WLAN, and/or WPAN transceivers) functioning in accordance with IEEE standards, 3GPP standards, or other standards, and configured to receive and transmit data via one or more external ports 654.

The server 615 may further include a user interface 662 configured to present information to a user and/or receive inputs from the user. As shown in FIG. 6, the user interface 662 may include a display screen 663 and I/O components 664 (e.g., ports, capacitive or resistive touch sensitive input panels, keys, buttons, lights, LEDs, external or built in keyboard). According to some embodiments, the user may access the server 615 via the user interface 662 to review information, make selections, and/or perform other functions.

In some embodiments, the server 615 may perform the functionalities as discussed herein as part of a “cloud” network or may otherwise communicate with other hardware or software components within the cloud to send, retrieve, or otherwise analyze data.

In general, a computer program product in accordance with an embodiment may include a computer usable storage medium (e.g., standard random access memory (RAM), an optical disc, a universal serial bus (USB) drive, or the like) having computer-readable program code embodied therein, wherein the computer-readable program code may be adapted to be executed by the processors 672, 659 (e.g., working in connection with the respective operating systems 679, 657) to facilitate the functions as described herein. In this regard, the program code may be implemented in any desired language, and may be implemented as machine code, assembly code, byte code, interpretable source code or the like (e.g., via Golang, Python, Scala, C, C++, Java, Actionscript, Objective-C, Javascript, CSS, XML). In some embodiments, the computer program product may be part of a cloud network of resources.

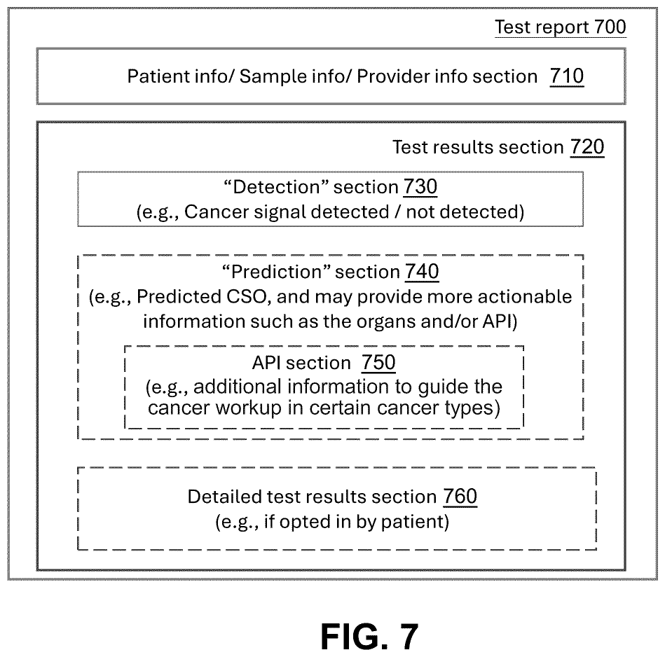

FIG. 7 is an example digital report 700 associated with a cancer screening test taken by an individual. According to embodiments, the digital report 700 may include a patient information section 710 that provides information associated with the individual (i.e., patient), test sample, and/or provider. Further, the digital report 700 may include a test results section 720 which may include a detection section 730, a prediction section 740, and a detailed test results section 760. According to embodiments, the detection section 730 may indicate whether a cancer signal is detected; the prediction section 740 may indicate a predicted CSO as well as include certain actionable information such as the organ(s) associated with the CSO and/or an API section 750 that may include additional information to guide any cancer workup in certain cancer types. In some embodiments, the detailed test results section may include additional information, for example, cancer-related risks and/or other information assessed using the data structure described herein, which may be included if the individual opts to receive this information. According to embodiments, the example digital report of FIG. 7 may have a single CSO, or a single CSO and a secondary CSO(s).

According to embodiments, the systems and methods may support additional or alternative functionalities associated with determining what information to include in a given digital report, as well as how the digital report is generated. In particular, the additional or alternative functionalities enable for uniquely conveying critical insights to individuals who take MCED tests, as well as healthcare providers, based on the detection of a cancer signal. When the MCED test identifies a cancer signal in a blood sample, the digital report may incorporate, in addition to a CSO prediction, one or more API categories that may be associated with or relevant to the CSO prediction. Accordingly, this functionality provides a more comprehensive understanding of the potential cancer type and its origin, thereby facilitating a more targeted diagnostic evaluation and aiding healthcare providers in making informed decisions regarding any next steps in the diagnostic process, and ultimately improving early and accurate cancer detection and treatment.

The systems and methods may initially determine, from a result of an MCED test, whether a cancer signal is detected in the individual's blood sample. If a cancer signal is detected, the systems and methods may identify any CSO prediction included in the result of the MCED test. Based on this CSO prediction, the systems and methods may employ a filtering or mapping mechanism to determine which, if any, API categories to include in the digital report.

Because the inclusion of a certain API category(ies) is contingent upon a specific CSO prediction, the digital report may be tailored to provide information that may be most relevant to the detected cancer signal. Further, this selection of APIs offers a nuanced perspective on the detected cancer signal, suggesting potential areas for further diagnostic evaluation.

For instance, if the CSO prediction is related to the cervix, the digital report may include API categories such as the HPV Associated Methylation Signal, Female Reproductive Tract Signal, and/or Neuroendocrine Signal. For further instance, if the CSO prediction pertains to the pancreas or gallbladder, the digital report may include API categories such as the Neuroendocrine Signal; if the CSO prediction pertains to the stomach or esophagus, the digital report may include API categories such as the Squamous Cell Signal and/or Neuroendocrine Signal; if the CSO prediction pertains to the hematopoietic and lymphoid organs, the digital report may include API categories such as the Lymphoid Lineage Signal, Myeloid Lineage, and/or Plasma Cell Lineage; and if CSO prediction pertains to the stomach or esophagus, the digital report may not include any API categories, which may reflect the specific nature of cancer signals originating from these tissues. It should be appreciated that different API categories may be included (or not included) depending on the different CSOs that may be identified in a given MCED test result.

According to embodiments, the systems and methods may map CSO predictions to API categories, such as based on research and analysis of different cancer types, their biomarkers, and molecular diagnostic information, e.g., associated methylation patterns. In embodiments, these mappings may be initially coded or determined. The systems and methods may store the mapping structure and reference the mapping structure in determining what information to include in a digital report. Further, the systems and methods may update the mapping to reflect the latest scientific discoveries and advancements in cancer detection technology. In operation, when a cancer signal is detected, the systems and methods may consult the mapping to determine which API categories, if any, should be included in the digital report based on the CSO prediction.

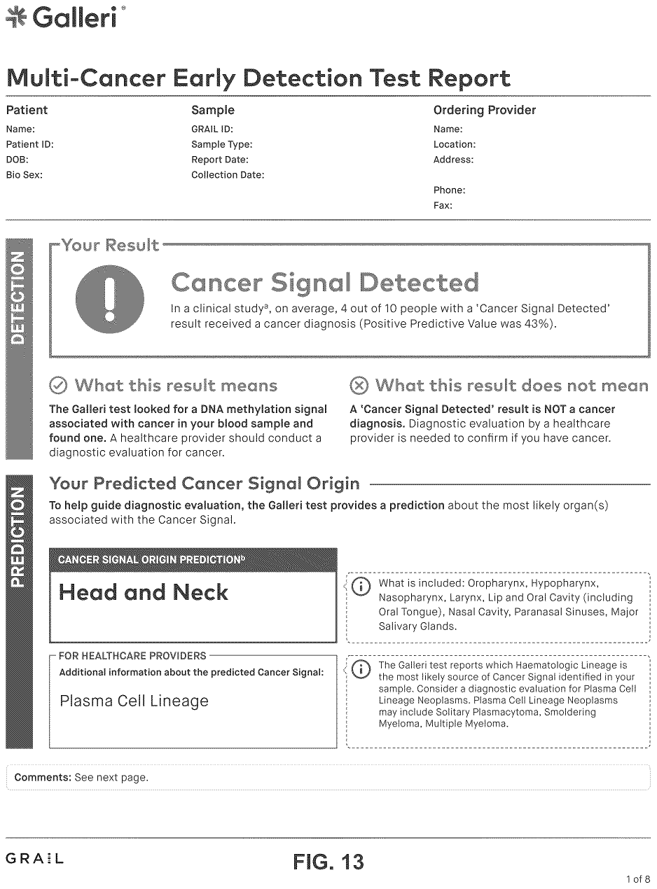

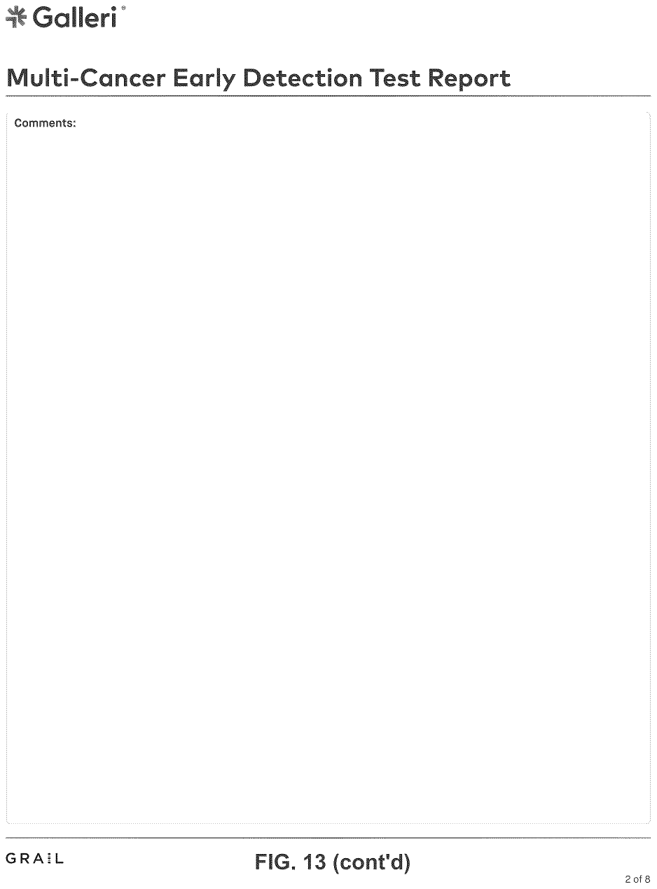

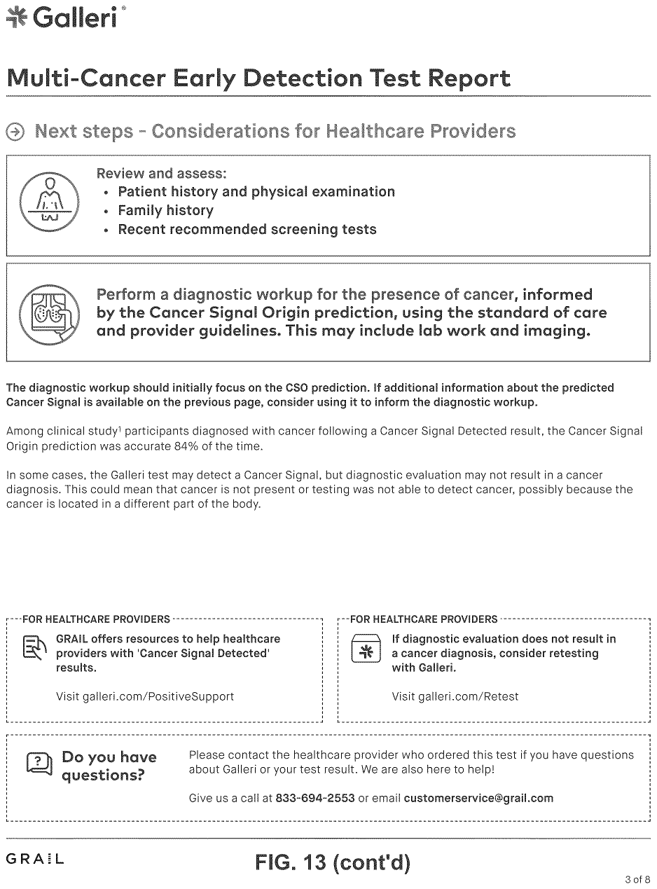

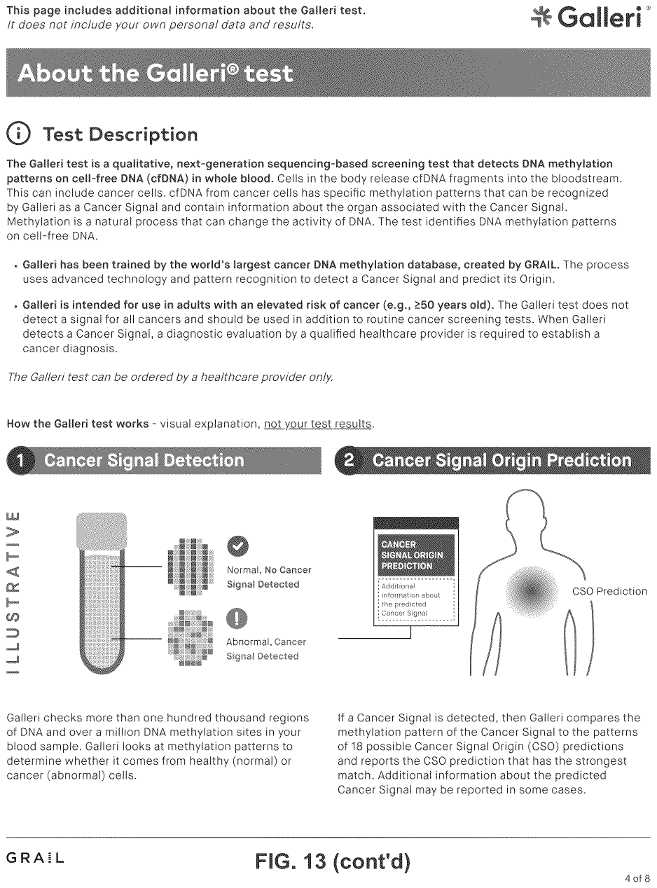

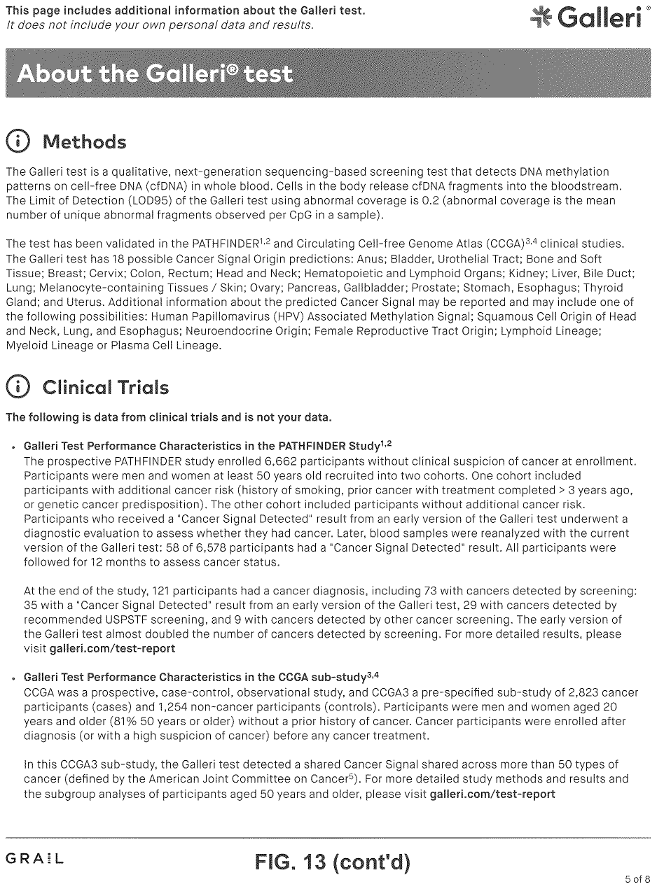

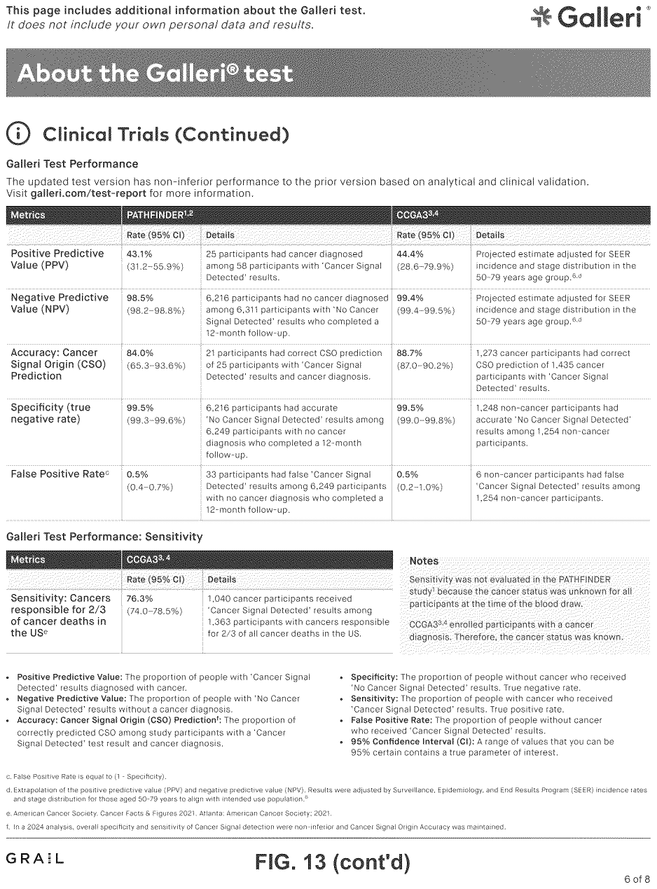

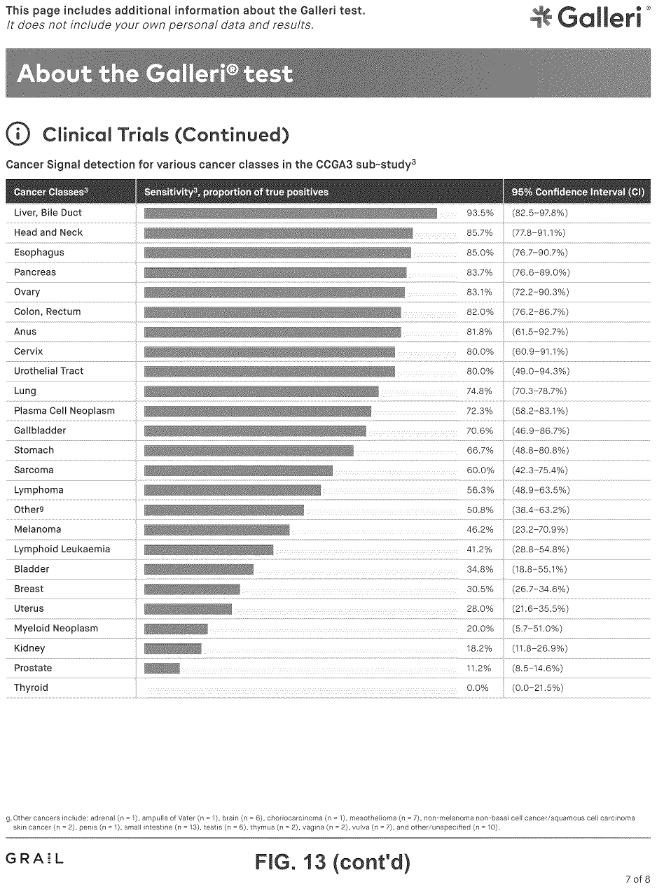

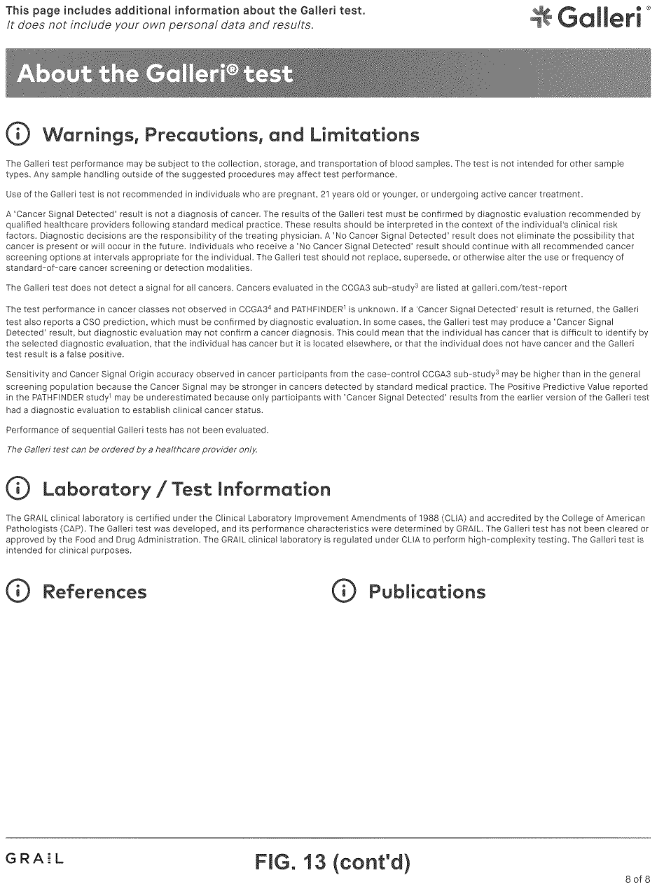

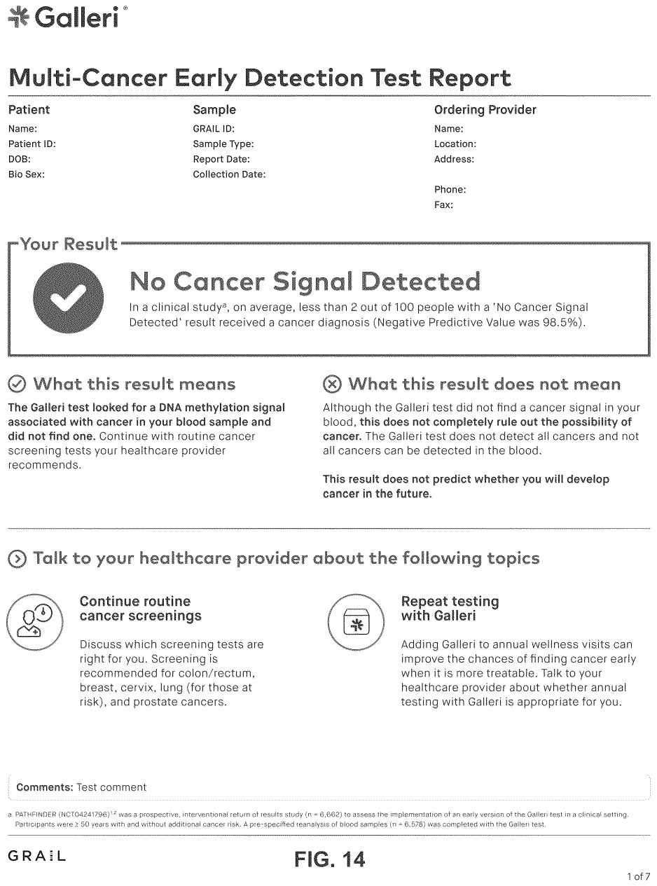

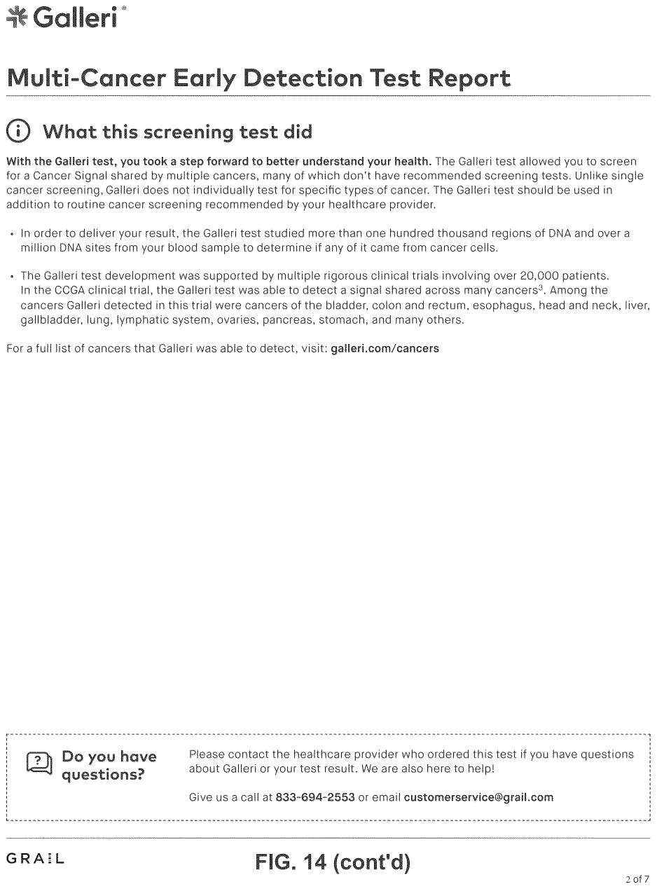

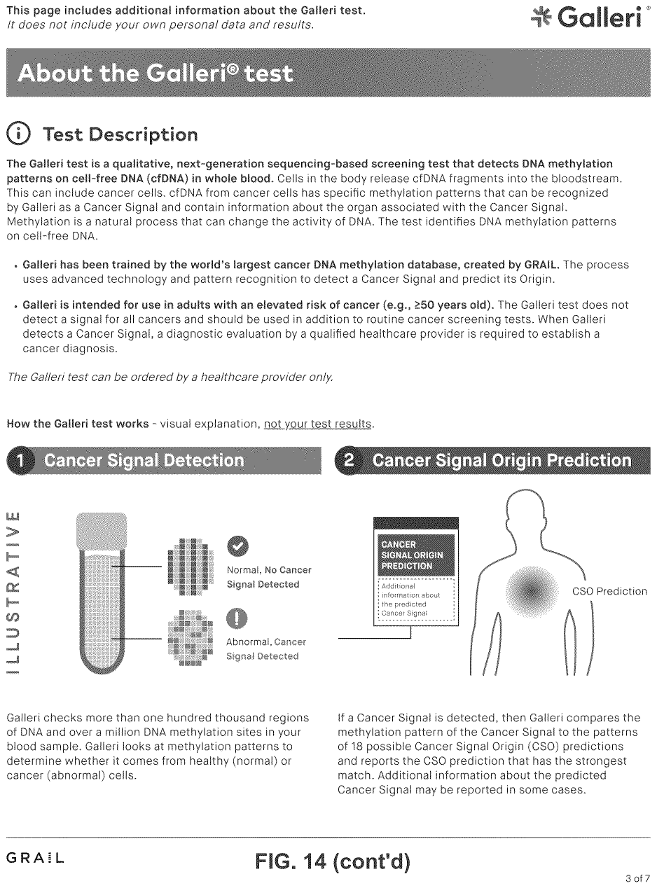

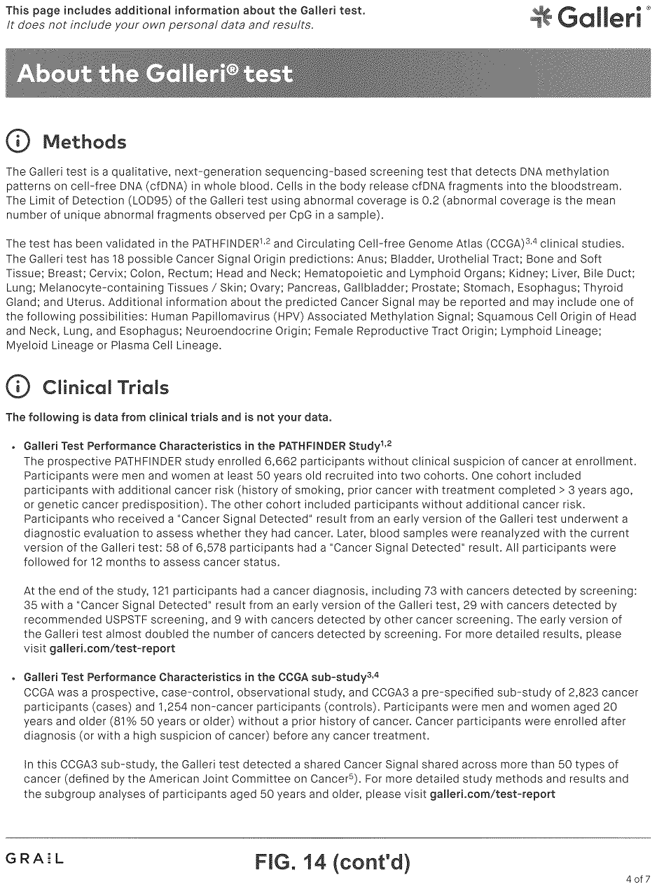

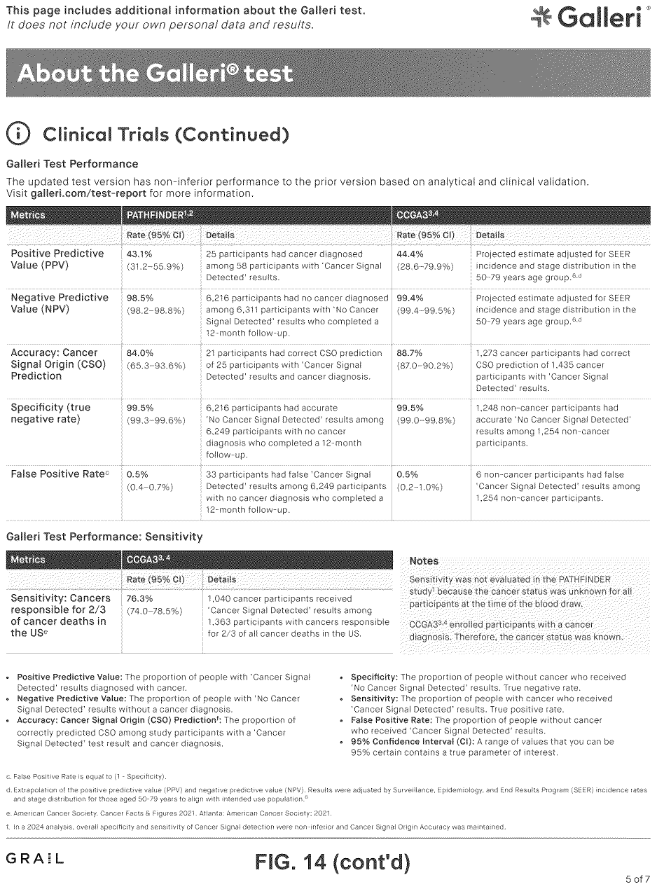

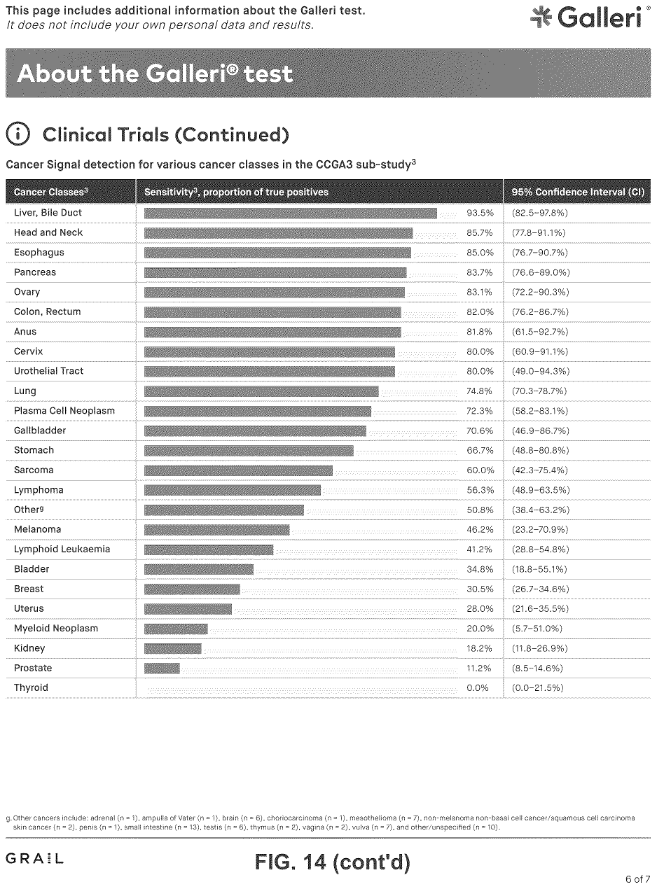

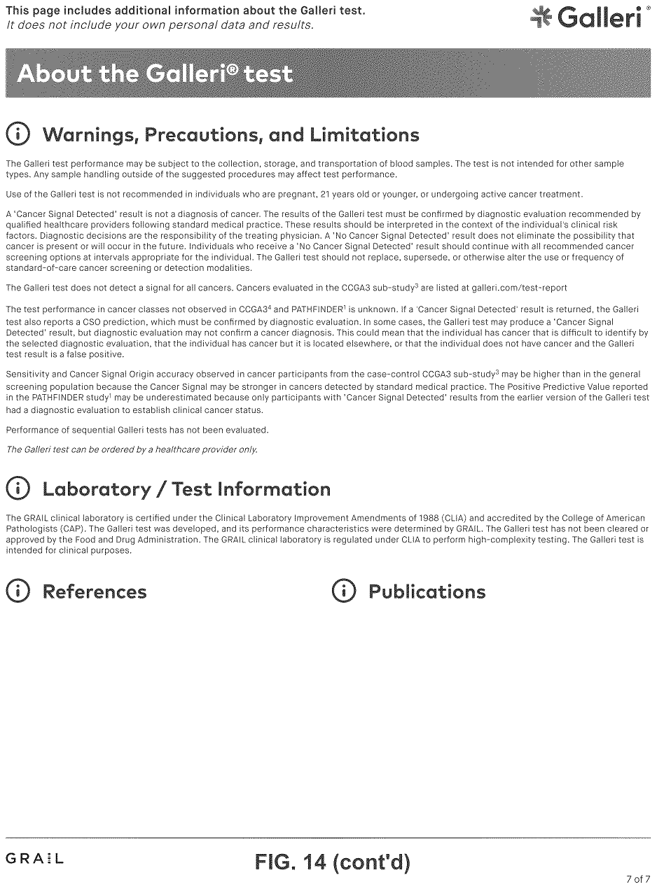

FIGS. 13 and 14 illustrate example digital reports providing information on cancer prediction results, in accordance with one or more embodiments. According to embodiments, the digital report provides a CSO prediction selected from a plurality of possible CSO predictions, e.g. 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more possible CSO predictions. In some embodiments, the possible CSO predictions include one or more of the following tissues of origin: Anus; Bladder, Urothelial Tract; Bone and Soft Tissue; Breast; Cervix; Colon, Rectum; Head and Neck; Hematopoietic and Lymphoid Organs; Kidney; Liver, Bile Duct; Lung; Melanocyte-containing Tissues/Skin; Ovary; Pancreas, Gallbladder; Prostate; Stomach, Esophagus; Thyroid; and Uterus.

In embodiments, if a CSO is detected, the corresponding digital report may include accompanying information about what is included in the CSO (e.g., a list of the most likely organs associated with the cancer signal). For example, the test report can indicate a CSO of “Head and Neck” being reported and list “What is included: Oropharynx, Hypopharynx, Nasopharynx, Larynx, Lip and Oral Cavity (including Oral Tongue), Nasal Cavity, Paranasal Sinuses, Major Salivary Glands.” Further, for example, the test report can indicate a CSO prediction of “Hematopoietic and Lymphoid Organs” and list “What is included: Bone Marrow, Primary and Secondary Lymphoid Tissue (Lymph nodes, Extranodal Lymphoid Tissue, Spleen, Thymus.”

Additionally, in embodiments, if a CSO is reported, then “additional predictive information” (API) may also be included in the digital report for certain CSO indications. As used herein, “additional predictive information” includes but is not limited to additional information relating to the biological origin of a given cancer signal, for instance, cellular lineage, viral origin (as applicable), and/or the like. For example, there may be 1, 2, 3, 4, 5, 6, 7, 8, 9, 10 or more categories of API. In some embodiments, the categories or API include one or more of the following categories: Human Papillomavirus (HPV) Associated Methylation Signal; Squamous Cell Signal of Head and Neck, Lung, and Esophagus; Neuroendocrine Signal; Female Reproductive Tract Signal; Lymphoid Lineage; and/or Myeloid Lineage or Plasma Cell Lineage.

FIGS. 13 & 14 also illustrate that the digital reports may include information associated with each of the API categories, which can provide a healthcare provider further information about what may happen next, where to look next, and/or other information. For example, for a test report that indicates a CSO prediction of Head and Neck, the API reported is “Human Papillomavirus (HPV) Associated Methylation Signal” and associated text for that API is, for example, “This is NOT a diagnosis of HPV. If the diagnostic evaluation based on the predicted Cancer Signal Origin does not lead to a cancer diagnosis, consider expanding the diagnostic evaluation to other organs possibly affected by HPV-associated cancers (Anus, Cervix, Head and Neck, Penis, Vagina, Vulva),” or the like.

For the API category “Squamous Cell Signal of Head and Neck, Lung, and Esophagus” the associated text can include, for example, “If the diagnostic evaluation based on the predicted Cancer Signal Origin does not lead to a cancer diagnosis, consider expanding the diagnostic evaluation to other organs possibly affected (Head and Neck, Lung, Esophagus),” or the like.

For the API category “Neuroendocrine Signal,” the associated text can include, for example, “If the diagnostic evaluation based on the predicted Cancer Signal Origin does not lead to a cancer diagnosis, consider expanding the diagnostic evaluation to other organs possibly affected by Low-or High-grade Neuroendocrine Tumors, which can affect most organs and body regions, frequently Lung and Gastrointestinal Tract,” or the like.

For the API category “Female Reproductive Tract Signal,” the associated text can include, for example, “If the diagnostic evaluation based on the predicted Cancer Signal Origin does not lead to a cancer diagnosis, consider expanding the diagnostic evaluation to other organs possibly affected (Ovary, Uterus, Cervix),” or the like.

For the API category “Lymphoid Lineage,” the associated text can include, for example, “The Galleri test reports which Hematologic Lineage is the most likely source of Cancer Signal identified in your sample. Consider a diagnostic evaluation for Lymphoid Linage Neoplasms. Lymphoid Lineage Neoplasms include Hodgkin Lymphoma and non-Hodgkin Lymphomas of B-or T-cells and can arise in primary or secondary Lymphoid organs, or Bone Marrow,” or the like.

For the API category “Myeloid Lineage” the associated text can include, for example, “The Galleri test reports which Hematologic Lineage is the most likely source of Cancer Signal identified in your sample. Consider a diagnostic evaluation for Myeloid Lineage Neoplasms. Myeloid Lineage Neoplasms include acute and chronic Myeloproliferative Neoplasms,” or the like.

For the API category “Plasma Cell Lineage,” the associated text can include, for example, “The Galleri test reports which Hematologic Lineage is the most likely source of Cancer Signal identified in your sample. Consider a diagnostic evaluation for Plasma Cell Lineage Neoplasms. Plasma Cell Lineage Neoplasms may include Solitary Plasmacytoma, Smoldering Myeloma, Multiple Myeloma,” or the like.

Further, in embodiments and for blood-related API categories (e.g. plasma cell lineage, lymphoid lineage, myeloid lineage), the additional information may not be reflexive. For example, the additional information may start with, for example, “The test reports which Hematologic Lineage is the most likely source of Cancer Signal identified in your sample. Consider a diagnostic evaluation for . . . [listing of what to consider upfront],” or the like. For the other APIs, the information may be additional or alternative, for example by stating “If the diagnostic evaluation based on the predicted Cancer Signal Origin does not lead to a cancer diagnosis, consider expanding the diagnostic evaluation to other organs possibly affected [listing of other organs],” or the like.

Further, in embodiments, if a CSO is reported, the digital report may indicate that further diagnostic steps may be needed. For example, in FIG. 13, the digital report indicates that “A Cancer Signal Detected result is NOT a cancer diagnosis. Diagnostic evaluation by a healthcare provider is needed to confirm if you have cancer.” Further, if no CSO is reported, the digital report may indicate that the individual may still have a cancer or a risk of cancer. For example, in FIG. 14, the digital report indicates that “Although the test did not find a Cancer Signal in your blood, this does not rule out the possibility of cancer. The test does not detect all cancers and not all cancers can be detected in the blood.”

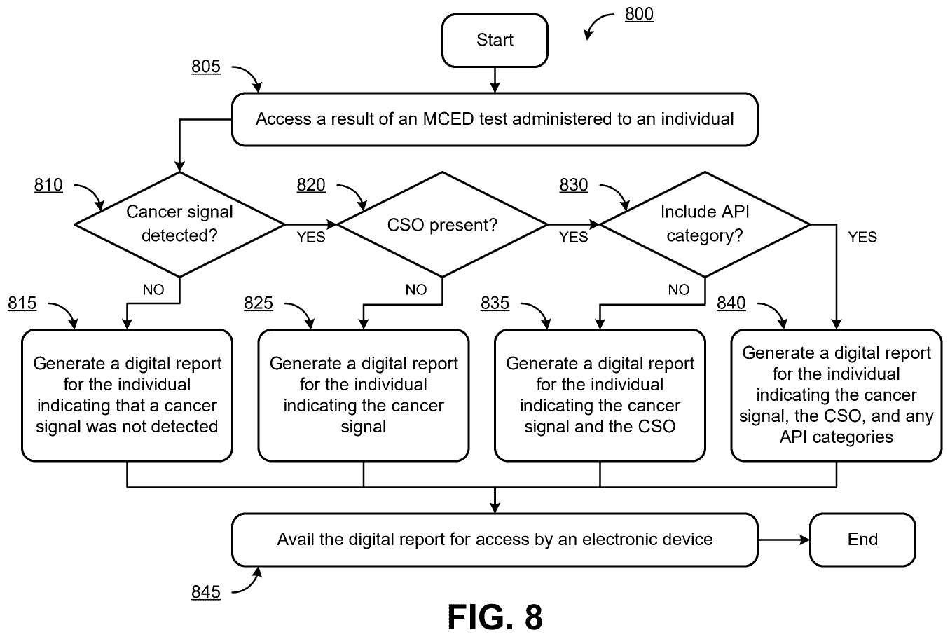

FIG. 8 is a block diagram of an example method 800 of generating a digital report resulting from a MCED test administered to an individual. The method 800 may be facilitated by one or more electronic devices (such as the server computer 115 as described with respect to FIG. 1).

The method 800 may begin when the electronic device accesses or obtains (block 805) a result of an MCED test administered to an individual. The electronic device may determine (block 810) whether the result of the MCED test indicates the detection of a cancer signal. If the result of the MCED test does not indicate the detection of a cancer signal (“NO”), the electronic device may generate (block 815) a digital report for the individual indicating that a cancer signal was not detected. If the result of the MCED test does indicate the detection of a cancer signal (“YES”), processing may proceed to block 820.

At block 820, the electronic device may determine whether a CSO is present in the result of the MCED test. If the CSO is not present (“NO”), the electronic device may generate (block 825) a digital report for the individual indicating the cancer signal. If the CSO is present (“YES”), processing may proceed to block 830.

At block 830, the electronic device may determine whether to include any API category(ies). In embodiments, the electronic device may perform this determination by referencing a stored mapping that maps certain CSOs to certain API categories. If the electronic device determines to not include any API categories (“NO”), the electronic device may generate (block 835) a digital report for the individual indicating the cancer signal and the CSO. If the electronic device determines to include an API category(ies) (“YES”), the electronic device may generate (block 840) a digital report for the individual indicating the cancer signal, the CSO, and an API category(ies) associated with the CSO.

The electronic device may avail (block 845) the digital report for access by an electronic device. In embodiments, the electronic device may be associated with the individual who initially took the MCED test, a clinician who is treating the individual, or another user.

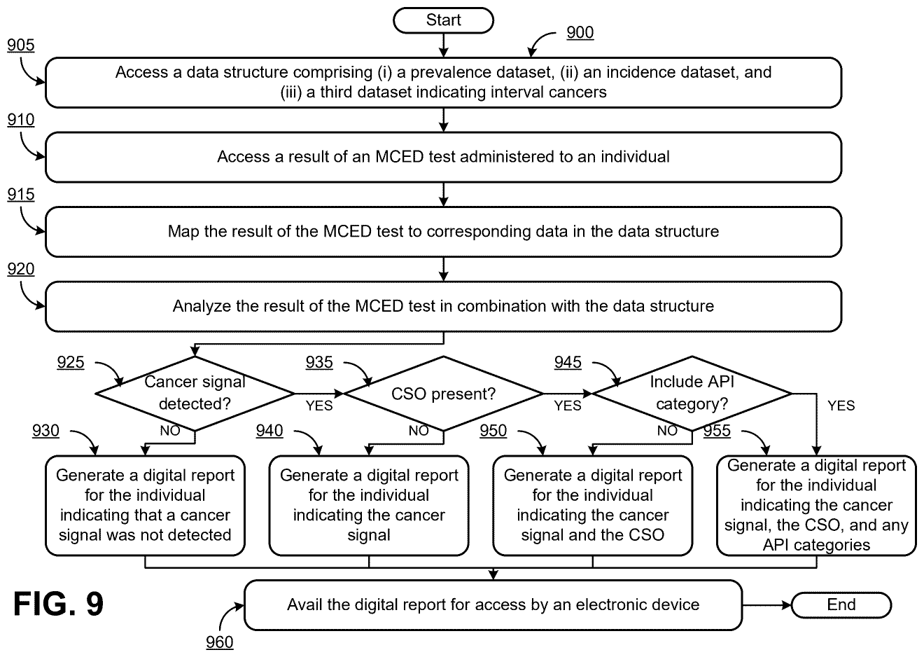

FIG. 9 is a block diagram of an example method 900 of generating a digital report resulting from a MCED test administered to an individual. The method 900 may be facilitated by one or more electronic devices (such as the server computer 115 as described with respect to FIG. 1).

The method 900 may begin when the electronic device accesses (block 905) a data structure that may include (i) a prevalence dataset indicating an initial prevalence of a plurality of individuals who have one or more of a plurality of cancers, (ii) an incidence dataset indicating an incidence of the plurality of individuals who are newly diagnosed with one or more of the plurality of cancers within a specified time period, (iii) a third dataset indicating instances of the plurality of individuals being diagnosed with one or more of the plurality of cancers between screening tests, or some combination thereof.

The electronic device may access or obtain (block 910) a result of the MCED test administered to the individual. The electronic device may map (block 915) the result of the MCED test to the corresponding data structure. In embodiments, the electronic device may map an age and gender of the individual to corresponding data within the data structure, resulting in a set of mapped data. It should be appreciated that the electronic device may map alternative or additional demographic factors and/or clinical characteristics.

The electronic device may analyze (block 920) the result of the MCED test in combination with the data structure (e.g., in the form of the set of mapped data) to determine a set of risk levels of the individual developing one or more of the plurality of cancers over the specified time period, wherein the set of risk levels accounts for at least the age and the gender of the individual. In embodiments, each risk level in the set of risk levels may be a percentage likelihood, a category, or another type of risk indication.

The electronic device may determine (block 925) whether the result of the MCED test indicates the detection of a cancer signal. If the result of the MCED test does not indicate the detection of a cancer signal (“NO”), the electronic device may generate (block 930) a digital report for the individual indicating that a cancer signal was not detected. If the result of the MCED test does indicate the detection of a cancer signal (“YES”), processing may proceed to block 935.

At block 935, the electronic device may determine whether a CSO is present in the result of the MCED test. If the CSO is not present (“NO”), the electronic device may generate (block 940) a digital report for the individual indicating the cancer signal. If the CSO is present (“YES”), processing may proceed to block 945.

At block 945, the electronic device may determine whether to include any API category(ies). In embodiments, the electronic device may perform this determination by referencing a stored mapping that maps certain CSOs to certain API categories. If the electronic device determines to not include any API categories (“NO”), the electronic device may generate (block 950) a digital report for the individual indicating the cancer signal and the CSO. If the electronic device determines to include an API category(ies) (“YES”), the electronic device may generate (block 955) a digital report for the individual indicating the cancer signal, the CSO, and an API category(ies) associated with the CSO.

It should be appreciated that the digital report generated at any of blocks 930, 940, 950, and 955 may additionally include any information that results from the analysis of block 920, including any information related to a set of risk levels, such as that described with respect to 232, 530, and 535, for example.

The electronic device may avail (block 960) the digital report for access by an electronic device. In embodiments, the electronic device may be associated with the individual who initially took the MCED test, a clinician who is treating the individual, or another user.

In some embodiments, a system leverages a population-level repository of cancer-related statistics to inform individualized inference and decision support. The repository may contain curated datasets spanning incidence, prevalence, stage distribution, progression rates, survival outcomes, and treatment response metrics across diverse demographic strata, including age, sex, ancestry, geographic region, and comorbidity profiles. The system can further incorporate multi-omic reference distributions (e.g., genomic, epigenomic, transcriptomic, proteomic) and imaging-derived phenotypes, normalized and harmonized across cohorts to mitigate batch effects and ascertain comparability. Statistical summaries and priors may be maintained at multiple resolutions (e.g., cancer-type, tissue-of-origin, risk factor combinations) and can be periodically recalibrated using incoming real-world evidence to reflect shifts in practice patterns and screening uptake. Privacy-preserving analytics, such as de-identification and federated aggregation, can be used to guard subject confidentiality while enabling robust population stratification. The repository may expose inference-ready features, calibration curves, and operating points that the prediction model can utilize in real time or near-real time.

In one aspect, the system applies a cancer prediction workflow to estimate a likelihood that a test sample obtained from an individual is associated with a cancer signal. The workflow can ingest sample metadata and assay-derived features produced by laboratory instruments, including but not limited to signals from circulating analytes such as cell-free nucleic acid patterns, protein panels, or other molecular and biophysical signatures. The workflow may include automated quality checks, feature transformation and harmonization, and an inference module comprising one or more probabilistic or machine-learned models that output an overall cancer-likelihood score, optional tissue-of-origin probabilities, and uncertainty measures. The system can record chain-of-custody and laboratory information system identifiers to ensure sample integrity and enable traceable audit. Model selection and parameters may be versioned, and performance can be monitored using holdout controls and synthetic references to maintain consistency over time. The output can be expressed along with confidence intervals and model provenance to facilitate downstream decisions.

The cancer prediction model may be a machine-learning model trained on a training dataset of samples obtained from individuals, some with cancer diagnoses and some without cancer diagnoses. In some embodiments, the training datasets may be further partitioned based on demographic information, cancer type, cancer stage, other contextual factors, etc. The machine-learning model may take the form of linear models, decision trees, random forests, gradient-boosted trees, support vector machines, k-nearest neighbors, naive Bayes, Gaussian processes, multilayer perceptrons, convolutional neural networks, recurrent neural networks (LSTM/GRU), Transformers, graph neural networks, autoencoders, generative adversarial networks, reinforcement learning frameworks, hybrid architectures, etc. The cancer prediction model may be architected to include a plurality of submodels operating in unison to generate the cancer prediction. The cancer prediction may be binary, indicating a positive or a negative for detection of cancer signal. The cancer prediction may further indicate one or more likelihoods for tissues-of-origin for the detected cancer signal. For example, the cancer prediction may indicate that one cancer type has a highest likelihood based on the computational analyses performed on the sequencing data for the individual, with a second cancer type having a next highest likelihood based on the analyses. Example implementations of cancer prediction models that may be implemented in the cancer prediction workflow include U.S. Pat. No. 12,027,237 B2 entitled “ANOMALOUS FRAGMENT DETECTION AND CLASSIFICATION,” U.S. Publication No. 2020/0365229 A1 entitled “MODEL-BASED FEATURIZATION AND CLASSIFICATION,” U.S. Publication No. 2021/0125686 A1 entitled “CANCER CLASSIFICATION WITH TISSUE OF ORIGIN THRESHOLDING,” and U.S. Publication No 2024/0161867 A1 entitled “OPTIMIZATION OF MODEL-BASED FEATURIZATION AND CLASSIFICATION,” all of which are incorporated by reference.

In another aspect, the system screens the individual-level prediction against the population statistics to tailor prognosis estimation and diagnostic work-up recommendations. The prediction may be calibrated using stratum-specific priors derived from the repository to adjust post-test probability in light of relevant demographics, exposure history, and comorbid conditions. Thresholds for action can be dynamically selected to meet desired operating characteristics (e.g., sensitivity, specificity, positive predictive value) for a given population segment and care setting, with fairness and equity constraints applied to minimize disparate impact. The system may generate a work-up profile that prioritizes follow-on evaluations aligned with the most likely tissue-of-origin and stage distributions for the matched stratum, while accounting for contraindications and resource availability. Decision rationale and estimated net benefit can be surfaced to clinicians, and the system can log recommendations, overrides, and outcomes for continuous learning. This screening step enables context-aware triage that is harmonized with real-world risk baselines.

In further embodiments, based on the calibrated prediction and associated uncertainty, the system can queue a subsequent sampling from the individual for an additional, confirmatory, or orthogonal test. The healthcare provider may determine an appropriate temporal window for resampling that balances biomarker dynamics, clinical urgency, and patient convenience, and can generate electronic orders, reminders, and consent prompts through integrated health information systems. The system can allocate phlebotomy or collection resources, verify eligibility criteria, and coordinate logistics with laboratory capacity to reduce turnaround time. Business rules may escalate or defer resampling when quality metrics, borderline scores, or discordant findings meet predefined criteria. All actions can be recorded in an auditable workflow ledger, with notifications delivered to authorized care team members.

Additionally, the system can use the prediction to queue sequence processing or equivalent high-resolution analysis of a reserve sample previously collected and retained under appropriate chain-of-custody. Upon triggering conditions—such as high uncertainty, intermediate likelihood scores within a gray zone, or suspected tissue ambiguity—the orchestration engine can submit the reserve aliquot to a sequencing or measurement pipeline configured for deeper coverage, expanded panels, or alternative assay modalities. The system may manage batch assignment, instrument reservation, and run configuration, while maintaining model-to-assay compatibility via versioned analysis templates. Results from the reserve-sample analysis can be fused with the initial prediction using ensemble or Bayesian updating schemes, producing an updated likelihood and refined tissue-of-origin distribution along with reproducibility metrics. These operations support efficient secondary analysis without requiring immediate patient resampling, thus improving diagnostic yield and operational efficiency.

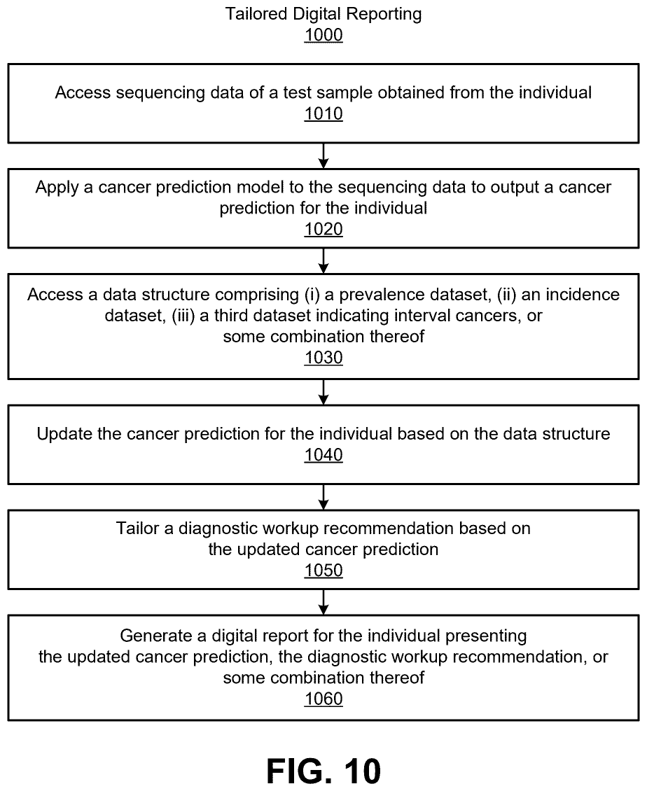

FIG. 10 illustrates a flow diagram describing tailored digital reporting 1000, in accordance with some embodiments. An analytics system may perform the process of tailored digital reporting 1000, which may be a computer-implemented method. In other embodiments, another computer system or device may perform some or all of the steps described. In other embodiments, the process of tailored digital reporting 1000 may include additional steps, fewer steps, different steps, or some combination thereof.

The analytics system initiates the tailored digital reporting 1000 workflow for an identified individual by creating a case record and binding it to a unique workflow session. The system may authenticate the requesting endpoint and/or retrieve configuration parameters associated with the requesting electronic device. The system may further access information relevant to the individual, e.g., including the individual's permission settings, prescreening questionnaire responses, prescreening results (e.g., performed by a healthcare provider), and other medical history. The analytics system may also access additional predictive information on the individual. The additional predictive information may include demographic factors and clinical characteristics, e.g., age, sex, relevant exposures, family history, recent screening history, cellular lineage, viral origin. The additional predictive information can be incorporated as covariates or as conditioning variables.

The analytics system accesses 1010 sequencing data of a test sample obtained from the individual by connecting to a laboratory information system and a secure object store where assay outputs are deposited. The analytics system may obtain raw and processed files (e.g., FASTQ, BAM/CRAM, VCF, methylation matrices, fragment length distributions) together with sample metadata, barcode identifiers, and quality control metrics such as read depth, duplication rate, contamination estimates, and coverage uniformity. The analytics system may perform preprocessing techniques on the sequencing data. The sequencing data may include data on genetic variants, methylome, DNA or RNA, etc.

The analytics system applies 1020 a cancer prediction model to the sequencing data to output a cancer prediction for the individual. The analytics system can extract features from the sequencing data for input into the cancer prediction model. Example features include variants (e.g., small variants, insertions, deletions, etc.), methylation/methylome signatures, copy-number patterns, and other analyte-specific vectors. The cancer prediction model may be a machine-learning model trained on a training dataset of samples obtained from individuals, some with cancer diagnoses and some without cancer diagnoses. The cancer prediction model may be architected to include a plurality of submodels operating in unison to generate the cancer prediction. The cancer prediction may be binary, indicating a positive or a negative for detection of cancer signal. The cancer prediction may further indicate one or more likelihoods for tissues-of-origin for the detected cancer signal. For example, the cancer prediction may indicate that one cancer type has a highest likelihood based on the computational analyses performed on the sequencing data for the individual, with a second cancer type having a next highest likelihood based on the analyses.

The analytics system accesses 1030 a data structure comprising (i) a prevalence dataset, (ii) an incidence dataset, (iii) a third dataset indicating interval cancers, or some combination thereof. The data structure is implemented as a stratified, queryable store that indexes rates by demographics, geography, care setting, screening modality, and temporal windows. Prevalence tables provide baseline disease presence across strata; incidence tables describe new case emergence over time; interval-cancer tables quantify cancers detected between scheduled screenings or shortly after negative screens, parameterized by modality and interval length.

The analytics system updates 1040 the cancer prediction for the individual based on the data structure. The analytics system may perform Bayesian or equivalent post-test probability adjustment that integrates the cancer prediction model output with stratum-specific prevalence and incidence priors based on the data structure. The analytics system can identify the strata for the individual, based on the individual's personal characteristics. The analytics system can leverage the prevalence data, the incidence data, and the cancer-interval data pertaining to the individual's strata to update the cancer prediction for the individual. When recent screening history is present, the system modulates the posterior using interval-cancer rates that reflect residual risk within the applicable follow-up window. Calibration curves specific to the assay and population segment are applied to correct for distributional shift, and uncertainty propagation is computed so that the adjusted score carries credible intervals reflecting both model and epidemiologic variance. The updated prediction includes an actionable category assignment (e.g., low, intermediate, high likelihood), tissue-of-origin refinements, and a record of the exact priors and calibration artifacts used in the update step. Following the example above, the analytics system may, based on the data structure, update the cancer prediction such that the second cancer type is more likely than the first cancer type.

The analytics system tailors 1050 a diagnostic workup recommendation, and/or a personalized next steps suggestion, based on the updated cancer prediction. The analytics system may store a decision tree mapping out different steps for the diagnostic workup based on the cancer prediction output by the cancer prediction model. The analytics system may prune branches of the decision tree based on the cancer prediction. For example, the decision tree may provide steps for the diagnostic workup for all the screened for cancer types. Upon identifying a highest likelihood cancer type as the tissue-of-origin of the positive detected cancer signal, the analytics system may prune all branches for other cancer types, leaving only steps for the highest likelihood cancer type. Or, in another example, the analytics system may prune branches for cancer types with predicted likelihood as the tissue-of-origin below some threshold. By mapping the individual's posterior risk and likely tissue-of-origin to guideline-anchored pathways and site-specific evaluation options, the analytics system tailors the diagnostic workup recommendation to the individual and the cancer prediction for that individual. The analytics system may further select the next steps, e.g., such as confirmatory laboratory assays, imaging, specialty referral, or observation, subject to contraindications, local resource availability, and provider preferences. The system may rank the candidate diagnostic workup steps by expected net clinical utility and turnaround considerations. Recommendations may further be contextualized with rationales. In some embodiments, the analytics system may tailor parameters of the steps, e.g., frequency, location of screening, types of screening tests, etc. In some examples, when the cancer prediction indicates that a cancer signal is detected or otherwise present, the analytics system can identify somatic and/or driver mutations (e.g. KRAS, EGFR) to guide therapy in patients/individuals with positive signal, or to help narrow diagnostics. Additionally and/or alternatively, the analytics system can also use added genomic data, quantitative metrics, and/or patient demographics to suggest next steps.

Further, in some examples, when the cancer prediction indicates that no cancer signal is detected or present (e.g., when the screening test does not include, indicate, or result in a CSO prediction), the analytics system can identify certain inherited germline variants that can guide the individual's risk assessment (e.g., BRCA 1/2, Lynch syndrome). Such results can be included in the individual's test report, for example reporting when certain genes or variants are “not detected” such that patients/individuals/providers can be informed that those genes or variants were considered and looked for. Further suggestions that can be reported for ‘no cancer signal detected’ results can also include quantitative metrics to indicate the strength of any cancer signals and/or a personalized risk score to help clinicians prioritize screening vigilance.

The analytics system generates 1060 a digital report for the individual presenting the updated cancer prediction, the diagnostic workup recommendation or personalized next steps suggestion, or some combination thereof. The analytics system assembles structured and narrative sections that include the adjusted likelihood score with confidence intervals, tissue-of-origin probabilities, calibration notes, and the recommended diagnostic pathway with reasoning and contingencies. The document is rendered as interoperable artifacts (e.g., FHIR resources, HL7 messages, signed PDFs) and localized as needed. Further contextual content can be included including summations of statistics identified from the data structure for the strata pertaining to the individual. The system delivers the report to designated endpoints, e.g., such as a healthcare provider's inbox, patient portal, or care-coordination platform.

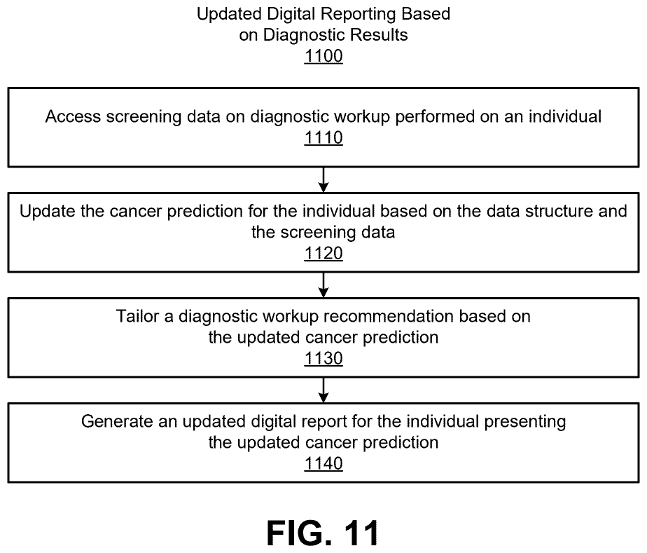

FIG. 11 illustrates a flow diagram describing updated tailored digital reporting 1100 based on diagnostic results, in accordance with some embodiments. The process of updated tailored digital reporting 1100 may follow thereafter tailored digital reporting 1000 in FIG. 10. For example, following an initial presentation of the digital report to a healthcare provider, the healthcare provider in consultation with the individual may perform a diagnostic workup, the results of which are provided to the analytics system for updating the cancer prediction. An analytics system may perform the process of updated tailored digital reporting 1100, which may be a computer-implemented method. In other embodiments, another computer system or device may perform some or all of the steps described. In other embodiments, the process of tailored digital reporting 1000 may include additional steps, fewer steps, different steps, or some combination thereof.

The analytics system accesses 1110 screening data on a diagnostic workup performed on an individual. The analytics system may establish secure connections to clinical systems that store recent test results, e.g., such as imaging findings, laboratory values, pathology reports, procedure notes, clinician notes or diagnoses. Retrieved artifacts are validated against expected schemas, de-identified or minimized per policy, and normalized into an internal timeline that captures test modality, collection time, performing site, key results, and associated quality flags. These harmonized screening data are linked to the individual's case record to support downstream analysis.

The analytics system updates 1120 the cancer prediction for the individual based on the data structure and the screening data by combining the prior model output with evidence from the newly-performed diagnostics. The analytics system accesses the data structure encoded with the prevalence data, the incidence data, and the interval-cancer data across different strata, and uses these as priors in a Bayesian or equivalent probabilistic update. Each screening observation is mapped to test-specific operating characteristics—such as sensitivity, specificity, and likelihood ratios—derived from assay performance databases and guideline summaries, enabling computation of post-test probabilities conditioned on the individual's demographics, comorbidities, and time since prior screening. The analytics system propagates uncertainty from both epidemiologic sources and measurement error and produces an updated cancer prediction. The updated cancer prediction may include an adjustment on likelihood of presence of cancer signal, an adjustment on likelihood for each of one or more cancer types, tissue-of-origin priority adjustments, or some combination thereof.

The analytics system tailors 1130 a diagnostic workup recommendation based on the updated cancer prediction by translating the posterior risk and evidence profile into context-aware next actions. In one or more embodiments, the analytics system may further modify the decision tree based on the screening data. For example, at the initial prediction, the analytics system identifies a first cancer type as the highest likelihood tissue-of-origin. Following the screening data that rules out that first cancer type, the analytics system may prune or otherwise downweight the branch pertaining to the first cancer type. The analytics system may then identify a second cancer type as the now likeliest tissue-of-origin, tailoring the diagnostic workup recommendations to the now likeliest tissue-of-origin. Thresholds for action are set to meet desired operating points for the matched population stratum and care setting, and fairness constraints are applied to avoid disparate treatment across subgroups. The system assembles order sets with preauthorization data, schedules, and preparatory instructions, ranks alternatives by expected net benefit and turnaround time, and annotates each recommendation with rationale referencing the updated risk, test performance, and applicable guidelines.

The analytics system generates 1140 an updated digital report for the individual presenting the updated cancer prediction by composing a structured document that integrates the recalibrated score, tissue-of-origin probabilities, confidence intervals, and the tailored workup plan. The report includes a decision rationale section that explains how recent diagnostics influenced the posterior risk, cites the epidemiologic datasets and performance references used, and provides actionable next steps with timing and contact details. The system renders the report and delivers it to designated endpoints including the clinician inbox, patient portal, and care coordination systems.

The analytics system may continue to iterate through the updated tailored digital reporting workflow 1100 as the report provides an updated tailored diagnostic workup recommendation to the healthcare provider. As the healthcare provider continues performing diagnostic tests to confirm or invalidate the computationally-based predictions, the analytics system may continue updating the cancer prediction and the recommended diagnostic workup actions to account for the real-time screening results.

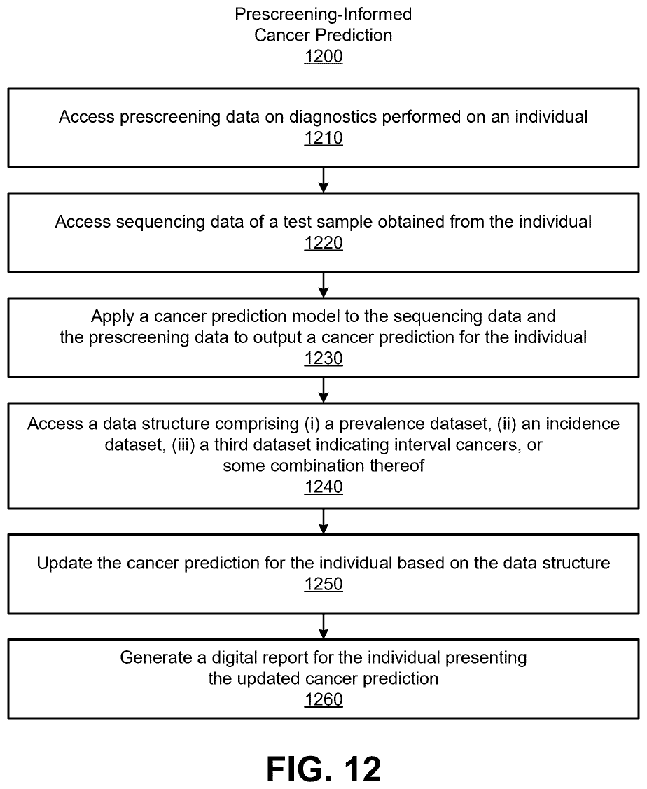

FIG. 12 illustrates a flow diagram describing prescreening-informed cancer prediction 1200, in accordance with some embodiments. An analytics system may perform the process of prescreening-informed cancer prediction 1200, which may be a computer-implemented method. In other embodiments, another computer system or device may perform some or all of the steps described. In other embodiments, the process of prescreening-informed cancer prediction 1200 may include additional steps, fewer steps, different steps, or some combination thereof.

The analytics system initiates the prescreening-informed cancer prediction 1200 workflow by creating a case record for the individual and binding it to a unique execution context. The analytics system loads configuration parameters associated with the ordering provider and laboratory, verifies consent and data-sharing authorizations, and resolves identifiers across internal and external systems. Orchestration services allocate compute resources, establish secure connections to clinical and laboratory repositories, and pin versions of models and reference datasets so that all subsequent outputs are reproducible.

The analytics system accesses 1210 prescreening data on diagnostics performed on an individual. The analytics system may access the prescreening data by querying EHR from secure databases, radiology, or laboratory information systems through standardized interfaces. Retrieved artifacts include structured test results (e.g., imaging categorizations, biomarker values, prior screen outcomes), procedure histories, and unstructured narratives from reports. The analytics system validates schemas, normalizes codes using controlled vocabularies, and applies natural language processing to extract salient features and uncertainty qualifiers from free text.

The analytics system accesses 1220 sequencing data of a test sample obtained from the individual by subscribing to laboratory data drops and pulling raw and processed files together with quality metrics. Inputs may include read files, alignment tracks, variant calls, methylation matrices, and fragment-length distributions, accompanied by barcodes, lot numbers, and instrument identifiers. Integrity checks (hash verification, schema validation) and quality assessments (coverage, contamination, duplication) are performed, and acceptable datasets are transformed into model-ready features via standardized pipelines. The system associates the sequencing payload with the case record.

The analytics system applies 1230 a cancer prediction model to the sequencing data and the prescreening data to output a cancer prediction for the individual. The analytics system may extract features from the sequencing data. Example features include variants (e.g., small variants, insertions, deletions, etc.), methylome signatures, copy-number patterns, and other analyte-specific vectors. The analytics system concatenates the sequencing features prescreening covariates that capture demographics, exposures, and recent test outcomes. In some embodiments, the analytics system may modify parameters in the cancer prediction model based on the prescreening data. In some embodiments, the cancer prediction model includes a plurality of models applied to differing contexts. Based on the prescreening data, the analytics system selects the appropriate model for inference on the sequencing data of the individual. For example, one model may be trained for individuals of the female sex above the age of 60. If the individual's demographics correspond to the model's target demographics, the analytics system selects that model for use in predicting the cancer prediction for the individual. The cancer prediction model may be a machine-learning model trained on a training dataset of samples obtained from individuals, some with cancer diagnoses and some without cancer diagnoses. The cancer prediction may be binary, indicating a positive or a negative for detection of cancer signal. The cancer prediction may further indicate one or more likelihoods for tissues-of-origin for the detected cancer signal. For example, the cancer prediction may indicate that one cancer type has a highest likelihood based on the computational analyses performed on the sequencing data for the individual, with a second cancer type having a next highest likelihood based on the analyses.

The analytics system accesses 1240 a data structure comprising (i) a prevalence dataset, (ii) an incidence dataset, (iii) a third dataset indicating interval cancers, or some combination thereof. The data structure is implemented as a stratified, queryable store indexed by attributes such as age, sex, geography, care setting, and screening modality. The system selects strata relevant to the individual's profile and recent screening timeline, caches the priors locally, and exposes them as inference-ready parameters for calibration and risk adjustment.

The analytics system updates 1250 the cancer prediction for the individual based on the data structure by integrating the model output with the selected epidemiologic priors. The analytics system performs Bayesian or equivalent post-test probability updates, modulating residual risk using interval-cancer rates when recent screens are present and adjusting for temporal factors such as time since last screening. Uncertainty from both model and population sources is propagated to compute credible intervals, and distribution-shift checks trigger correction curves when necessary. The resulting updated prediction includes an actionable risk category and refined tissue-of-origin probabilities, together with a complete audit trail of priors and transformation steps.

The analytics system generates 1260 a digital report for the individual presenting the updated cancer prediction. The analytics system may generate the report by composing structured and narrative sections that describe the adjusted likelihood, confidence bounds, and the rationale for the update. The report is rendered and delivered to designated endpoints, e.g., such as a healthcare provider's electronic device, a patient portal, or a care-coordination platform.

### Example Clauses

Clause 1. A non-transitory computer-readable storage medium storing: a digital report for presenting information on a cancer screening test for an individual, the presented information including (i) a cancer signal that was detected, (ii) a cancer signal of origin (CSO) for the cancer signal, and (iii) a set of additional predictive information (API), the digital report generated by: obtaining, by at least one computer processor, a result of the cancer screening test administered to the individual, wherein the result of the cancer screening test indicates (i) that the cancer signal was detected, and (ii) the CSO for the cancer signal; determining, by the at least one computer processor based on an inclusion of the CSO for the cancer signal within a stored mapping, a set of additional predictive information (API) to be included in the digital report; and generating, by the at least one computer processor, the digital report for the individual, wherein the digital report indicates (i) that the cancer signal was detected, (ii) the CSO for the cancer signal, and the set of API.

Clause 2. The non-transitory computer-readable storage medium of clause 1, wherein the cancer screening test is a multi-cancer early detection (MCED) test.

Clause 3. The non-transitory computer-readable storage medium of any one of clauses 1-2, wherein generating the digital report for the individual comprises: determining if the result of the cancer screening test indicates that the cancer signal was detected; and responsive to determining that the result of the cancer screening test indicates that the cancer signal was detected, generating the digital report to include the CSO for the cancer signal.

Clause 4. The non-transitory computer-readable storage medium of any one of clauses 1-3, wherein the API includes additional information relating to biological origin of the CSO for the cancer signal.

Clause 5. The non-transitory computer-readable storage medium of any one of clauses 1-4, wherein the API includes a set of one or more risk levels for other individuals of a population having a cancer diagnosis following the cancer screening test.

Clause 6. The non-transitory computer-readable storage medium of clause 5, wherein the set of one or more risk levels includes a general risk level of other individuals of a same age and a same gender of the individual having a cancer diagnosis, and an individual risk level of the individual being diagnosed with cancer over a specified time period.

Clause 7. A computer-implemented method comprising: obtaining, by at least one computer processor, a result of the cancer screening test administered to the individual, wherein the result of a cancer screening test indicates (i) that a cancer signal was detected, and (ii) a cancer signal origin (CSO) for the cancer signal; determining, by the at least one computer processor based on an inclusion of the CSO for the cancer signal within a stored mapping, a set of additional predictive information (API) to be included in the digital report; and generating, by the at least one computer processor, a digital report for the individual, wherein the digital report indicates (i) that the cancer signal was detected, (ii) the CSO for the cancer signal, and (iii) the set of API.

Clause 8. The computer-implemented method of clause 7, wherein the cancer screening test is a multi-cancer early detection (MCED) test.

Clause 9. The computer-implemented method of any one of clauses 7-8, wherein generating the digital report for the individual comprises: determining if the result of the cancer screening test indicates that the cancer signal was detected; and responsive to determining that the result of the cancer screening test indicates that the cancer signal was detected, generating the digital report to include the CSO for the cancer signal.

Clause 10. The computer-implemented method of any one of clauses 7-9, wherein the API includes additional information relating to biological origin of the CSO for the cancer signal.

Clause 11. The computer-implemented method of any one of clauses 7-10, wherein the API includes a set of one or more risk levels for other individuals of a population having a cancer diagnosis following the cancer screening test.

Clause 12. The computer-implemented method of clause 11, wherein the set of one or more risk levels includes a general risk level of other individuals of a same age and a same gender of the individual having a cancer diagnosis, and an individual risk level of the individual being diagnosed with cancer over a specified time period.

Clause 13. A non-transitory computer-readable storage medium storing: a digital report for presenting information on a cancer screening test for an individual, the presented information including (i) whether a cancer signal was detected, and (ii) a risk level that a screening test for cancer would result in the individual being diagnosed with that cancer, the digital report generated by: accessing a cancer prediction for the individual output by a cancer prediction model applied to sequencing data obtained from sequencing a test sample collected from the individual; accessing a data structure comprising a prevalence dataset, an incidence dataset, an interval-cancer dataset, or some combination thereof; updating the cancer prediction for the individual based on the data structure; tailoring a diagnostic workup recommendation inclusive of recommended steps for a healthcare provider based on the updated cancer prediction; and generating the digital report presenting the updated cancer prediction and the diagnostic workup recommendation tailored to the individual based on the updated cancer prediction.

Clause 14. The non-transitory computer-readable storage medium of clause 13, wherein the digital report is generated further by: accessing information relevant to the individual including additional predictive information, wherein updating the cancer prediction for the individual storing the additional predictive information as covariates or conditioning variables associated with a case record.

Clause 15. The non-transitory computer-readable storage medium of clause 14, wherein the API includes additional information relating to biological origin of the CSO for the cancer signal.

Clause 16. The non-transitory computer-readable storage medium of any one of clauses 13-15, wherein the data structure comprises stratifications for the additional predictive information, and wherein updating the cancer prediction for the individual based on the data structure comprises: identifying a stratum of the individual based on the additional predictive information; retrieving values in the prevalence dataset, values in the incidence dataset, or values in the interval-cancer dataset corresponding to the stratum of the individual; and modulating a posterior probability using the values in the prevalence dataset, the values in the incidence dataset, or the values in the interval-cancer dataset corresponding to the stratum of the individual to update the cancer prediction.

Clause 17. The non-transitory computer-readable storage medium of any one of clauses 13-16, wherein the prevalence dataset comprises information on an initial prevalence of a plurality of individuals who have one or more of a plurality of cancers.

Clause 18. The non-transitory computer-readable storage medium of clause 17, wherein the incidence dataset comprises information on an incidence of the plurality of individuals who are newly diagnosed with one or more of the plurality of cancers within a specified time period.

Clause 19. The non-transitory computer-readable storage medium of clause 17, wherein the interval-cancer dataset comprises information on instances of the plurality of individuals being diagnosed with one or more of the plurality of cancers between screening tests.

Clause 20. The non-transitory computer-readable storage medium of any one of clauses 13-19, wherein tailoring the diagnostic workup recommendation inclusive of the recommended steps for the healthcare provider based on the updated cancer prediction comprises: accessing a decision tree mapping diagnostic workup steps to cancer types; pruning branches of the decision tree based on the updated cancer prediction comprising likelihoods for the cancer types; and selecting the recommended steps from remaining branches of the decision tree.

Clause 21. The non-transitory computer-readable storage medium of clause 20, wherein pruning the branches of the decision tree comprises: pruning branches of the decision tree associated with one or more cancer types below a likelihood threshold.

Clause 22. The non-transitory computer-readable storage medium of any one of clauses 13-21, wherein the digital report is generated further by: accessing screening data on a diagnostic workup performed by a healthcare provider on the individual; reupdating the cancer prediction for the individual based on the data structure and the screening data; tailoring a subsequent diagnostic workup recommendation based on the reupdated cancer prediction; and updating the digital report for the individual presenting the reupdated cancer prediction and the subsequent diagnostic workup recommendation.

Clause 23. The non-transitory computer-readable storage medium of any one of clauses 13-22, wherein the cancer prediction for the individual is output by the cancer prediction model applied to the sequencing data and prescreening data on additional predictive information of the individual.

Clause 24. A computer-implemented method comprising: accessing a cancer prediction for the individual output by a cancer prediction model applied to sequencing data obtained from sequencing a test sample collected from the individual; accessing a data structure comprising a prevalence dataset, an incidence dataset, an interval-cancer dataset, or some combination thereof, updating the cancer prediction for the individual based on the data structure; tailoring a diagnostic workup recommendation inclusive of recommended steps for a healthcare provider based on the updated cancer prediction; generating a digital report presenting the updated cancer prediction and the diagnostic workup recommendation tailored to the individual based on the updated cancer prediction; and transmitting the digital report to a client device for presentation on an electronic display.

Clause 25. The computer-implemented method of clause 24, further comprising: accessing information relevant to the individual including additional predictive information, wherein updating the cancer prediction for the individual includes storing the additional predictive information as covariates or conditioning variables associated with a case record.

Clause 26. The computer-implemented method of clause 25, wherein the API includes additional information relating to biological origin of the CSO for the cancer signal.

Clause 27. The computer-implemented method of any one of clauses 24-26, wherein the data structure comprises stratifications for the additional predictive information, and wherein updating the cancer prediction for the individual based on the data structure comprises: identifying a stratum of the individual based on the additional predictive information; retrieving values in the prevalence dataset, values in the incidence dataset, or values in the interval-cancer dataset corresponding to the stratum of the individual; and modulating a posterior probability using the values in the prevalence dataset, the values in the incidence dataset, or the values in the interval-cancer dataset corresponding to the stratum of the individual to update the cancer prediction.

Clause 28. The computer-implemented method of any one of clauses 24-27, wherein the prevalence dataset comprises information on an initial prevalence of a plurality of individuals who have one or more of a plurality of cancers.

Clause 29. The computer-implemented method of clause 28, wherein the incidence dataset comprises information on an incidence of the plurality of individuals who are newly diagnosed with one or more of the plurality of cancers within a specified time period.

Clause 30. The computer-implemented method of clause 28, wherein the interval-cancer dataset comprises information on instances of the plurality of individuals being diagnosed with one or more of the plurality of cancers between screening tests.

Clause 31. The computer-implemented method of any one of clauses 24-30, wherein tailoring the diagnostic workup recommendation inclusive of the recommended steps for the healthcare provider based on the updated cancer prediction comprises: accessing a decision tree mapping diagnostic workup steps to cancer types; pruning branches of the decision tree based on the updated cancer prediction comprising likelihoods for the cancer types; and selecting the recommended steps from remaining branches of the decision tree.

Clause 32. The computer-implemented method of clause 31, wherein pruning the branches of the decision tree comprises: pruning branches of the decision tree associated with one or more cancer types below a likelihood threshold.

Clause 33. The computer-implemented method of any one of clauses 24-32, further comprising: accessing screening data on a diagnostic workup performed by a healthcare provider on the individual; reupdating the cancer prediction for the individual based on the data structure and the screening data; tailoring a subsequent diagnostic workup recommendation based on the reupdated cancer prediction; and updating the digital report for the individual presenting the reupdated cancer prediction and the subsequent diagnostic workup recommendation.

Clause 34. The computer-implemented method of any one of clauses 24-33, wherein the cancer prediction for the individual is output by the cancer prediction model applied to the sequencing data and prescreening data on additional predictive information of the individual.

Clause 35. A computer-implemented method of ascertaining cancer-related risks resulting from a cancer screening test administered to an individual using population-level classification data, the computer-implemented method comprising: accessing, by at least one computer processor, a data structure comprising (i) a prevalence dataset indicating an initial prevalence of a plurality of individuals who have one or more of a plurality of cancers, (ii) an incidence dataset indicating an incidence of the plurality of individuals who are newly diagnosed with one or more of the plurality of cancers within a specified time period, and (iii) a third dataset indicating instances of the plurality of individuals being diagnosed with one or more of the plurality of cancers between screening tests; obtaining, by the at least one computer processor, a result of the cancer screening test administered to the individual, wherein the result of the cancer screening test indicates whether a cancer signal was detected; analyzing, by the at least one computer processor, the result of the cancer screening test in combination with the data structure to determine a set of risk levels of the individual developing one or more of the plurality of cancers over the specified time period, wherein the set of risk levels accounts for at least an age and a gender of the individual; and generating, by the at least one computer processor based on the set of risk levels, a digital report for the individual, wherein the digital report indicates (i) whether a cancer signal was detected, (ii) if a cancer signal was not detected, a risk level, of the set of risk levels, of the individual being diagnosed with at least one cancer, of the plurality of cancers, over the specified time period, and (iii) if a cancer signal was detected, and for each of at least some of the cancers of the plurality of cancers, an additional risk level, of the set of risk levels, that a screening test corresponding to that cancer would result in the individual being diagnosed with that cancer.

Clause 36. The computer-implemented method of clause 35, wherein the cancer screening test is a multi-cancer early detection (MCED) test, and wherein if the result of the cancer screening test indicates that the cancer signal was detected, the result of the cancer screening test further indicates a cancer signal origin (CSO) for the cancer signal.

Clause 37. The computer-implemented method of any one of clauses 35-36, wherein each risk level of the set of risk levels is a percentage likelihood.

Clause 38. The computer-implemented method of any one of clauses 35-37, wherein each risk level of the set of risk levels is a category.

Clause 39. The computer-implemented method of any one of clauses 35-38, wherein generating the digital report for the individual comprises: generating, by the at least one computer processor based on the set of risk levels, the digital report for the individual, wherein the digital report indicates (i) that the cancer signal was not detected, (ii) a general risk level of another individual of the age and the gender of the individual having cancer, (iii) an individual risk level of the individual being diagnosed with the at least one cancer, of the plurality of cancers, over the specified time period.

Clause 40. The computer-implemented method of any one of clauses 35-39, wherein generating the digital report for the individual comprises: generating, by the at least one computer processor based on the set of risk levels, the digital report for the individual, wherein the digital report indicates (i) that the cancer signal was not detected, (ii) a plurality of general risk levels of another individual of the age and the gender of the individual respectively having the plurality of cancers, (iii) a plurality of individual risk levels of the individual respectively being diagnosed with the plurality of cancers over the specified time period.

Clause 41. The computer-implemented method of any one of clauses 35-40, wherein generating the digital report for the individual comprises: generating, by the at least one computer processor based on the set of risk levels, the digital report for the individual, wherein the digital report indicates (i) that the cancer signal was detected, (ii) for each of the plurality of cancers, a pre-screen risk level of another individual of the age and the gender of the individual being diagnosed with that cancer absent screening, (iii) for each of the plurality of cancers, a prevalent screen risk level of the individual being diagnosed with that cancer over the specified time period, and (iv) for each of the plurality of cancers, an incident screen risk level of the individual being diagnosed with that cancer over another time period subsequent to the specified time period.

Clause 42. The computer-implemented method of any one of clauses 35-41, wherein generating the digital report for the individual comprises: generating, by the at least one computer processor based on the set of risk levels, the digital report for the individual, wherein the digital report indicates (i) that the cancer signal was detected, (ii) a cancer signal origin (CSO) for the cancer signal, (iii) for each of the plurality of cancers, a pre-screen risk level of another individual of the age and the gender of the individual being diagnosed with that cancer absent screening, (iv) for each of the plurality of cancers, a prevalent screen risk level of the individual being diagnosed with that cancer over the specified time period, and (v) for each of the plurality of cancers, an incident screen risk level of the individual being diagnosed with that cancer over another time period subsequent to the specified time period.

Clause 43. The computer-implemented method of any one of clauses 35-42, wherein analyzing the result of the cancer screening test in combination with the data structure comprises: mapping the result of the cancer screening test, and the age and the gender of the individual to corresponding data within the data structure, resulting in a set of mapped data; and analyzing, by the at least one computer processor, the set of mapped data to determine the set of risk levels of the individual developing one or more of the plurality of cancers over the specified time period.

Clause 44. A system for ascertaining cancer-related risks resulting from a cancer screening test administered to an individual using population-level classification data, comprising: a memory storing a set of computer-readable instructions and a data structure comprising (i) a prevalence dataset indicating an initial prevalence of a plurality of individuals who have one or more of a plurality of cancers, (ii) an incidence dataset indicating an incidence of the plurality of individuals who are newly diagnosed with one or more of the plurality of cancers within a specified time period, and (iii) a third dataset indicating instances of the plurality of individuals being diagnosed with one or more of the plurality of cancers between screening tests; and one or more processors interfaced with the memory, and configured to execute the set of computer-readable instructions to cause the one or more processors to: obtain a result of the cancer screening test administered to the individual, wherein the result of the MCED test indicates whether a cancer signal was detected, analyze the result of the cancer screening test in combination with the data structure to determine a set of risk levels of the individual developing one or more of the plurality of cancers over the specified time period, wherein the set of risk levels accounts for at least an age and a gender of the individual, and generate, based on the set of risk levels, a digital report for the individual, wherein the digital report indicates (i) whether a cancer signal was detected, (ii) if a cancer signal was not detected, a risk level, of the set of risk levels, of the individual being diagnosed with at least one cancer, of the plurality of cancers, over the specified time period, and (iii) if a cancer signal was detected, and for each of at least some of the cancers of the plurality of cancers, an additional risk level, of the set of risk levels, that a screening test corresponding to that cancer would result in the individual being diagnosed with that cancer.

Clause 45. The system of clause 44, wherein the cancer screening test is a multi-cancer early detection (MCED) test, and wherein if the result of the cancer screening test indicates that the cancer signal was detected, the result of the cancer screening test further indicates a cancer signal origin (CSO) for the cancer signal.

Clause 46. The system of any one of clauses 44-45, wherein each risk level of the set of risk levels is a percentage likelihood.

Clause 47. The system of any one of clauses 44-46, wherein each risk level of the set of risk levels is a category.

Clause 48. The system of any one of clauses 44-47, wherein the digital report indicates (i) that the cancer signal was not detected, (ii) a general risk level of another individual of the age and the gender of the individual having cancer, (iii) an individual risk level of the individual being diagnosed with the at least one cancer, of the plurality of cancers, over the specified time period.

Clause 49. The system of any one of clauses 44-48, wherein the digital report indicates (i) that the cancer signal was not detected, (ii) a plurality of general risk levels of another individual of the age and the gender of the individual respectively having the plurality of cancers, (iii) a plurality of individual risk levels of the individual respectively being diagnosed with the plurality of cancers over the specified time period.

Clause 50. The system of any one of clauses 44-49, wherein the digital report indicates (i) that the cancer signal was detected, (ii) for each of the plurality of cancers, a pre-screen risk level of another individual of the age and the gender of the individual being diagnosed with that cancer absent screening, (iii) for each of the plurality of cancers, a prevalent screen risk level of the individual being diagnosed with that cancer over the specified time period, and (iv) for each of the plurality of cancers, an incident screen risk level of the individual being diagnosed with that cancer over another time period subsequent to the specified time period.

Clause 51. The system of any one of clauses 44-50, wherein the digital report indicates (i) that the cancer signal was detected, (ii) a cancer signal origin (CSO) for the cancer signal, (iii) for each of the plurality of cancers, a pre-screen risk level of another individual of the age and the gender of the individual being diagnosed with that cancer absent screening, (iv) for each of the plurality of cancers, a prevalent screen risk level of the individual being diagnosed with that cancer over the specified time period, and (v) for each of the plurality of cancers, an incident screen risk level of the individual being diagnosed with that cancer over another time period subsequent to the specified time period.

Clause 52. The system of any one of clauses 44-51, wherein to analyze the result of the cancer screening test in combination with the data structure, the one or more processors are configured to execute the set of computer-readable instructions to cause the one or more processors to: map the result of the cancer screening test, and the age and the gender of the individual to corresponding data within the data structure, resulting in a set of mapped data, and analyze the set of mapped data to determine the set of risk levels of the individual developing one or more of the plurality of cancers over the specified time period.

Clause 53. A non-transitory computer-readable storage medium configured to store instructions executable by one or more processors, the instructions comprising: instructions for accessing a data structure comprising (i) a prevalence dataset indicating an initial prevalence of a plurality of individuals who have one or more of a plurality of cancers, (ii) an incidence dataset indicating an incidence of the plurality of individuals who are newly diagnosed with one or more of the plurality of cancers within a specified time period, and (iii) a third dataset indicating instances of the plurality of individuals being diagnosed with one or more of the plurality of cancers between screening tests; instructions for obtaining a result of a cancer screening test administered to an individual, wherein the result of the cancer screening test indicates whether a cancer signal was detected; instructions for analyzing the result of the cancer screening test in combination with the data structure to determine a set of risk levels of the individual developing one or more of the plurality of cancers over the specified time period, wherein the set of risk levels accounts for at least an age and a gender of the individual; and instructions for generating, based on the set of risk levels, a digital report for the individual, wherein the digital report indicates (i) whether a cancer signal was detected, (ii) if a cancer signal was not detected, a risk level, of the set of risk levels, of the individual being diagnosed with at least one cancer, of the plurality of cancers, over the specified time period, and (iii) if a cancer signal was detected, and for each of at least some of the cancers of the plurality of cancers, an additional risk level, of the set of risk levels, that a screening test corresponding to that cancer would result in the individual being diagnosed with that cancer.

Clause 54. The non-transitory computer-readable storage medium of clause 53, wherein the cancer screening test is a multi-cancer early detection (MCED) test, and wherein if the result of the cancer screening test indicates that the cancer signal was detected, the result of the MCED test further indicates a cancer signal origin (CSO) for the cancer signal.

Clause 55. A computer-implemented method in an electronic device of rendering a digital report for an individual related to a result of a cancer screening test administered to the individual, the computer-implemented method comprising: receiving, from a server computer by at least one computer processor of the electronic device, the digital report for the individual, wherein the result of the cancer screening test was analyzed in combination with a data structure to determine a set of risk levels, accounting for at least an age and a gender of the individual, of the individual developing one or more of a plurality of cancers over a specified time period, and wherein the data structure comprises (i) a prevalence dataset indicating an initial prevalence of a plurality of individuals who have one or more of the plurality of cancers, (ii) an incidence dataset indicating an incidence of the plurality of individuals who are newly diagnosed with one or more of the plurality of cancers within the specified time period, and (iii) a third dataset indicating instances of the plurality of individuals being diagnosed with one or more of the plurality of cancers between screening tests; initiating, by the electronic device, an application executed by the at least one computer processor and configured to render the digital report; and rendering the digital report via a user interface of the electronic device and within an interface associated with the application, wherein the digital report that was rendered indicates (i) the result of the cancer screening test administered to the individual, and (ii) the set of risk levels.

Clause 56. The computer-implemented method of clause 55, wherein rendering the digital report comprises: rendering the digital report via the user interface of the electronic device and within the interface associated with the application, wherein the digital report that was rendered indicates (i) whether a cancer signal from the cancer screening test was detected, (ii) if a cancer signal was not detected, a risk level, of the set of risk levels, of the individual being diagnosed with at least one cancer, of the plurality of cancers, over the specified time period, and (iii) if a cancer signal was detected, and for each of at least some of the cancers of the plurality of cancers, an additional risk level, of the set of risk levels, that a screening test corresponding to that cancer would result in the individual being diagnosed with that cancer.

Clause 57. The computer-implemented method of any one of clauses 55-56, wherein the application is a web browser, and wherein the interface is a webpage.

Clause 58. The computer-implemented method of any one of clauses 55-57, wherein initiating the application configured to render the digital report comprises: receiving, via the user interface, a user input; and in response to receiving the user input, initiating the application.

Clause 59. The computer implemented method of any one of clauses 55-58, wherein rendering the digital report via the user interface of the electronic device and within the interface associated with the application comprises: receiving, via the user interface, a user input; and in response to receiving the user input, rendering the digital report.

Clause 60. A computer-implemented method in an electronic device of rendering a digital report for an individual related to a result of a cancer screening test administered to the individual, the computer-implemented method comprising: receiving, from a server computer by at least one computer processor of the electronic device, the digital report for the individual, wherein the result of the cancer screening test was analyzed in combination with a data structure to determine a set of risk levels, accounting for at least an age and a gender of the individual, of the individual developing one or more of a plurality of cancers over a specified time period, and wherein the data structure comprises (i) a prevalence dataset indicating an initial prevalence of a plurality of individuals who have one or more of the plurality of cancers, (ii) an incidence dataset indicating an incidence of the plurality of individuals who are newly diagnosed with one or more of the plurality of cancers within the specified time period, and (iii) a third dataset indicating instances of the plurality of individuals being diagnosed with one or more of the plurality of cancers between screening tests; initiating, by the electronic device, an application executed by the at least one computer processor and configured to render the digital report; and rendering the digital report via a user interface of the electronic device and within an interface associated with the application, wherein the digital report that was rendered indicates (i) that a cancer signal from the cancer screening test was detected, and (ii) a cancer signal origin (CSO) for the cancer signal.

Clause 61. The computer-implemented method of clause 60, wherein rendering the digital report comprises: rendering the digital report via the user interface of the electronic device and within the interface associated with the application, wherein the digital report that was rendered indicates (i) that the cancer signal from the cancer screening test was detected, (ii) the cancer signal origin (CSO) for the cancer signal, and (iii) a set of additional predictive information (API) associated with the CSO.

Clause 62. The computer-implemented method of any one of clauses 60-61, wherein rendering the digital report comprises: rendering the digital report via the user interface of the electronic device and within the interface associated with the application, wherein the digital report that was rendered indicates (i) that the cancer signal from the cancer screening test was detected, (ii) the cancer signal origin (CSO) for the cancer signal, and (iii) the set of additional predictive information (API) associated with the CSO, and (iv) a set of detailed test results associated with the cancer screening test.

Clause 63. A computer-implemented method of generating a digital report resulting from a cancer screening test administered to an individual, the computer-implemented method comprising: obtaining, by at least one computer processor, a result of the cancer screening test administered to the individual, wherein the result of the cancer screening test indicates (i) that a cancer signal was detected, and (ii) a cancer signal origin (CSO) for the cancer signal; determining, by the at least one computer processor based on an inclusion of the CSO for the cancer signal within a stored mapping, a set of additional predictive information (API) to be included in the digital report; and generating, by the at least one computer processor, the digital report for the individual, wherein the digital report indicates (i) that the cancer signal was detected, (ii) the CSO for the cancer signal, and (iii) the set of API.

Clause 64. The computer-implemented method of clause 63, wherein the result of the cancer screening test was analyzed in combination with a data structure to determine a set of risk levels, accounting for at least an age and a gender of the individual, of the individual developing one or more of a plurality of cancers over a specified time period, and wherein the data structure comprises (i) a prevalence dataset indicating an initial prevalence of a plurality of individuals who have one or more of the plurality of cancers, (ii) an incidence dataset indicating an incidence of the plurality of individuals who are newly diagnosed with one or more of the plurality of cancers within the specified time period, and (iii) a third dataset indicating instances of the plurality of individuals being diagnosed with one or more of the plurality of cancers between screening tests.

Clause 65. The computer-implemented method of any one of clauses 63-64, wherein the set of additional predictive information (API) is included in the digital report.

Clause 66. A computer-implemented method of generating a digital report resulting from a cancer screening test administered to an individual, the computer-implemented method comprising: obtaining, by at least one computer processor, a result of the cancer screening test administered to the individual, wherein the result of the cancer screening test indicates that a cancer signal was not detected, wherein the result of the cancer screening test was analyzed in combination with a data structure to determine a set of risk levels, accounting for at least an age and a gender of the individual, of the individual developing one or more of a plurality of cancers over a specified time period, and wherein the data structure comprises (i) a prevalence dataset indicating an initial prevalence of a plurality of individuals who have one or more of the plurality of cancers, (ii) an incidence dataset indicating an incidence of the plurality of individuals who are newly diagnosed with one or more of the plurality of cancers within the specified time period, and (iii) a third dataset indicating instances of the plurality of individuals being diagnosed with one or more of the plurality of cancers between screening tests; and generating, by the at least one computer processor, the digital report for the individual, wherein the digital report indicates that a cancer signal was not detected.

### Additional Embodiments

Although the preceding and following text sets forth a detailed description of numerous different embodiments, it should be understood that the legal scope of the invention may be defined by the words of the claims set forth at the end of this patent. The detailed description is to be construed as exemplary only and does not describe every possible embodiment, as describing every possible embodiment would be impractical, if not impossible. One could implement numerous alternate embodiments, using either current technology or technology developed after the filing date of this patent, which would still fall within the scope of the claims.

Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the subject matter herein.

Additionally, certain embodiments are described herein as including logic or a number of routines, subroutines, applications, or instructions. These may constitute either software (e.g., code embodied on a non-transitory, machine-readable medium) or hardware. In hardware, the routines, etc., are tangible units capable of performing certain operations and may be configured or arranged in a certain manner. In example embodiments, one or more computer systems (e.g., a standalone, client or server computer system) or one or more hardware modules of a computer system (e.g., a processor or a group of processors) may be configured by software (e.g., an application or application portion) as a hardware module that operates to perform certain operations as described herein.

In various embodiments, a hardware module may be implemented mechanically or electronically. For example, a hardware module may comprise dedicated circuitry or logic that may be permanently configured (e.g., as a special-purpose processor, such as a field programmable gate array (FPGA) or an application-specific integrated circuit (ASIC)) to perform certain operations. A hardware module may also comprise programmable logic or circuitry (e.g., as encompassed within a general-purpose processor or other programmable processor) that may be temporarily configured by software to perform certain operations. It will be appreciated that the decision to implement a hardware module mechanically, in dedicated and permanently configured circuitry, or in temporarily configured circuitry (e.g., configured by software) may be driven by cost and time considerations.

Accordingly, the term “hardware module” should be understood to encompass a tangible entity, be that an entity that is physically constructed, permanently configured (e.g., hardwired), or temporarily configured (e.g., programmed) to operate in a certain manner or to perform certain operations described herein. Considering embodiments in which hardware modules are temporarily configured (e.g., programmed), each of the hardware modules need not be configured or instantiated at any one instance in time. For example, where the hardware modules comprise a general-purpose processor configured using software, the general-purpose processor may be configured as respective different hardware modules at different times. Software may accordingly configure a processor, for example, to constitute a particular hardware module at one instance of time and to constitute a different hardware module at a different instance of time.

Hardware modules may provide information to, and receive information from, other hardware modules. Accordingly, the described hardware modules may be regarded as being communicatively coupled. Where multiple of such hardware modules exist contemporaneously, communications may be achieved through signal transmission (e.g., over appropriate circuits and buses) that connect the hardware modules. In embodiments in which multiple hardware modules are configured or instantiated at different times, communications between such hardware modules may be achieved, for example, through the storage and retrieval of information in memory structures to which the multiple hardware modules have access. For example, one hardware module may perform an operation and store the output of that operation in a memory device to which it may be communicatively coupled. A further hardware module may then, at a later time, access the memory device to retrieve and process the stored output. Hardware modules may also initiate communications with input or output devices, and may operate on a resource (e.g., a collection of information).

The various operations of example methods described herein may be performed, at least partially, by one or more processors that are temporarily configured (e.g., by software) or permanently configured to perform the relevant operations. Whether temporarily or permanently configured, such processors may constitute processor-implemented modules that operate to perform one or more operations or functions. The modules referred to herein may, in some example embodiments, comprise processor-implemented modules.

Similarly, the methods or routines described herein may be at least partially processor-implemented. For example, at least some of the operations of a method may be performed by one or more processors or processor-implemented hardware modules. The performance of certain of the operations may be distributed among the one or more processors, not only residing within a single machine, but deployed across a number of machines. In some example embodiments, the processor or processors may be located in a single location (e.g., within a home environment, an office environment, or as a server farm), while in other embodiments the processors may be distributed across a number of locations.

The performance of certain of the operations may be distributed among the one or more processors, not only residing within a single machine, but deployed across a number of machines. In some example embodiments, the one or more processors or processor-implemented modules may be located in a single geographic location (e.g., within a home environment, an office environment, or a server farm). In other example embodiments, the one or more processors or processor-implemented modules may be distributed across a number of geographic locations.

Unless specifically stated otherwise, discussions herein using words such as “processing,” “computing,” “calculating,” “determining,” “presenting,” “displaying,” or the like may refer to actions or processes of a machine (e.g., a computer) that manipulates or transforms data represented as physical (e.g., electronic, magnetic, or optical) quantities within one or more memories (e.g., volatile memory, non-volatile memory, or a combination thereof), registers, or other machine components that receive, store, transmit, or display information.

As used herein any reference to “one embodiment” or “an embodiment” means that a particular element, feature, structure, or characteristic described in connection with the embodiment may be included in at least one embodiment. The appearances of the phrase “in one embodiment” in various places in the specification are not necessarily all referring to the same embodiment.

As used herein, the terms “comprises,” “comprising,” “may include,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

In addition, use of the “a” or “an” are employed to describe elements and components of the embodiments herein. This is done merely for convenience and to give a general sense of the description. This description, and the claims that follow, should be read to include one or at least one and the singular also may include the plural unless it is obvious that it is meant otherwise.

This detailed description is to be construed as examples and does not describe every possible embodiment, as describing every possible embodiment would be impractical.

1. A non-transitory computer-readable storage medium storing:
a digital report for presenting information on a cancer screening test for an individual, the presented information including (i) a cancer signal that was detected, (ii) a cancer signal of origin (CSO) for the cancer signal, and (iii) a set of additional predictive information (API), the digital report generated by:
obtaining, by at least one computer processor, a result of the cancer screening test administered to the individual, wherein the result of the cancer screening test indicates (i) that the cancer signal was detected, and (ii) the CSO for the cancer signal;
determining, by the at least one computer processor based on an inclusion of the CSO for the cancer signal within a stored mapping, a set of additional predictive information (API) to be included in the digital report; and
generating, by the at least one computer processor, the digital report for the individual, wherein the digital report indicates (i) that the cancer signal was detected, (ii) the CSO for the cancer signal, and the set of API.

2. The non-transitory computer-readable storage medium of claim 1, wherein the cancer screening test is a multi-cancer early detection (MCED) test.

3. The non-transitory computer-readable storage medium of claim 1, wherein generating the digital report for the individual comprises:
determining if the result of the cancer screening test indicates that the cancer signal was detected; and
responsive to determining that the result of the cancer screening test indicates that the cancer signal was detected, generating the digital report to include the CSO for the cancer signal.

4. The non-transitory computer-readable storage medium of claim 1, wherein the API includes additional information relating to biological origin of the CSO for the cancer signal.

5. The non-transitory computer-readable storage medium of claim 1, wherein the API includes a set of one or more risk levels for other individuals of a population having a cancer diagnosis following the cancer screening test.

6. The non-transitory computer-readable storage medium of claim 5, wherein the set of one or more risk levels includes a general risk level of other individuals of a same age and a same gender of the individual having a cancer diagnosis, and an individual risk level of the individual being diagnosed with cancer over a specified time period.

7. A computer-implemented method comprising:
obtaining, by at least one computer processor, a result of the cancer screening test administered to the individual, wherein the result of a cancer screening test indicates (i) that a cancer signal was detected, and (ii) a cancer signal origin (CSO) for the cancer signal;
determining, by the at least one computer processor based on an inclusion of the CSO for the cancer signal within a stored mapping, a set of additional predictive information (API) to be included in the digital report; and
generating, by the at least one computer processor, a digital report for the individual, wherein the digital report indicates (i) that the cancer signal was detected, (ii) the CSO for the cancer signal, and (iii) the set of API.

8. The computer-implemented method of claim 7, wherein the cancer screening test is a multi-cancer early detection (MCED) test.

9. The computer-implemented method of claim 7, wherein generating the digital report for the individual comprises:
determining if the result of the cancer screening test indicates that the cancer signal was detected; and
responsive to determining that the result of the cancer screening test indicates that the cancer signal was detected, generating the digital report to include the CSO for the cancer signal.

10. The computer-implemented method of claim 7, wherein the API includes additional information relating to biological origin of the CSO for the cancer signal.

11. The computer-implemented method of claim 7, wherein the API includes a set of one or more risk levels for other individuals of a population having a cancer diagnosis following the cancer screening test.

12. The computer-implemented method of claim 11, wherein the set of one or more risk levels includes a general risk level of other individuals of a same age and a same gender of the individual having a cancer diagnosis, and an individual risk level of the individual being diagnosed with cancer over a specified time period.

13. A non-transitory computer-readable storage medium storing:
a digital report for presenting information on a cancer screening test for an individual, the presented information including (i) whether a cancer signal was detected, and (ii) a risk level that a screening test for cancer would result in the individual being diagnosed with that cancer, the digital report generated by:
accessing a cancer prediction for the individual output by a cancer prediction model applied to sequencing data obtained from sequencing a test sample collected from the individual;
accessing a data structure comprising a prevalence dataset, an incidence dataset, an interval-cancer dataset, or some combination thereof;
updating the cancer prediction for the individual based on the data structure;
tailoring a diagnostic workup recommendation inclusive of recommended steps for a healthcare provider based on the updated cancer prediction; and
generating the digital report presenting the updated cancer prediction and the diagnostic workup recommendation tailored to the individual based on the updated cancer prediction.

14. The non-transitory computer-readable storage medium of claim 13, wherein the digital report is generated further by:
accessing information relevant to the individual including additional predictive information,
wherein updating the cancer prediction for the individual storing the additional predictive information as covariates or conditioning variables associated with a case record.

15. The non-transitory computer-readable storage medium of claim 14, wherein the API includes additional information relating to biological origin of the CSO for the cancer signal.

16. The non-transitory computer-readable storage medium of claim 13, wherein the data structure comprises stratifications for the additional predictive information, and wherein updating the cancer prediction for the individual based on the data structure comprises:
identifying a stratum of the individual based on the additional predictive information;
retrieving values in the prevalence dataset, values in the incidence dataset, or values in the interval-cancer dataset corresponding to the stratum of the individual; and
modulating a posterior probability using the values in the prevalence dataset, the values in the incidence dataset, or the values in the interval-cancer dataset corresponding to the stratum of the individual to update the cancer prediction.

17. The non-transitory computer-readable storage medium of claim 13, wherein the prevalence dataset comprises information on an initial prevalence of a plurality of individuals who have one or more of a plurality of cancers.

18. The non-transitory computer-readable storage medium of claim 17, wherein the incidence dataset comprises information on an incidence of the plurality of individuals who are newly diagnosed with one or more of the plurality of cancers within a specified time period.

19. The non-transitory computer-readable storage medium of claim 17, wherein the interval-cancer dataset comprises information on instances of the plurality of individuals being diagnosed with one or more of the plurality of cancers between screening tests.

20. The non-transitory computer-readable storage medium of claim 13, wherein tailoring the diagnostic workup recommendation inclusive of the recommended steps for the healthcare provider based on the updated cancer prediction comprises:
accessing a decision tree mapping diagnostic workup steps to cancer types;
pruning branches of the decision tree based on the updated cancer prediction comprising likelihoods for the cancer types; and
selecting the recommended steps from remaining branches of the decision tree.

21. The non-transitory computer-readable storage medium of claim 20, wherein pruning the branches of the decision tree comprises:
pruning branches of the decision tree associated with one or more cancer types below a likelihood threshold.

22. The non-transitory computer-readable storage medium of claim 13, wherein the digital report is generated further by:
accessing screening data on a diagnostic workup performed by a healthcare provider on the individual;
reupdating the cancer prediction for the individual based on the data structure and the screening data;
tailoring a subsequent diagnostic workup recommendation based on the reupdated cancer prediction; and
updating the digital report for the individual presenting the reupdated cancer prediction and the subsequent diagnostic workup recommendation.

23. The non-transitory computer-readable storage medium of claim 13, wherein the cancer prediction for the individual is output by the cancer prediction model applied to the sequencing data and prescreening data on additional predictive information of the individual.

24-66. (canceled)
